(19) United States
(12) Patent Application Publication
(10) Pub. No.: US 2010/0048411 A1
(43) Pub. Date: Feb. 25, 2010

(54) SUBSTRATE FOR THE GROWTH OF CULTURED CELLS IN THREE DIMENSIONS

(75) Inventors: Stefan Alexander Przyborski, Durham (GB); Neil Cameron, Durham (GB)

Correspondence Address: SPECKMAN LAW GROUP PLLC, 1201 THIRD AVENUE, SUITE 330, SEATTLE, WA, 98101, US

(73) Assignee: REINNERVATE LIMITED, Durham (GB)

(21) Appl. No.: 12/298,742

(22) PCT Filed: Apr. 24, 2007
(86) PCT No.: PCT/GB07/01464

§ 371 Date: May 7, 2009

(30) Foreign Application Priority Data
Apr. 28, 2006 (GB) 0608403.2
May 20, 2006 (GB) 0610120.8
Jan. 12, 2007 (GB) 0700592.9

(51) Int. Cl. C12N 5/07; C12Q 1/02; C40B 30/04; C08J 9/28; C12N 5/071; C12M 3/00

(52) U.S. Cl. 506/9; 435/395; 435/401; 435/29; 521/65; 435/377; 435/289.1; 435/1.1

(57) ABSTRACT

We describe a cell culture substrate comprising a polymerised high internal phase emulsion polymer adapted and modified for use in the routine culture of cells in three dimensions; typically mammalian cells and the use of the substrate in a cell culture system for investigation and analysis of proliferation, differentiation and function of cells.

The invention relates to a cell culture substrate comprising a polymerised high internal phase emulsion polymer (polyHIPE) adapted for installation and use in existing cell culture plastic-ware for the growth of cells, typically mammalian cells and the use of the substrate in a cell culture system for analysis of proliferation, differentiation and function of cells.

The culturing of eukaryotic cells, for example mammalian cells, has become a routine procedure and cell culture conditions which allow cells to proliferate, differentiate and function are well defined. Typically, cell culture of mammalian cells requires a sterile vessel, usually manufactured from plastics (typically polystyrene), defined growth medium and, in some examples, feeder cells and serum, typically calf serum. The feeder cells function to provide signals which stimulate cell proliferation and/or maintain cells in an undifferentiated state and can influence cell function. The culturing of prokaryotic cells, for example bacterial cells is also an established technique and has been used for many years for the production of valuable molecules.

The culturing of mammalian cells has many applications and there are numerous in vitro assays and models where cell culture is used for experimentation and research; for example the use of cells in tissue engineering; the use of mammalian expression systems for the production of recombinant protein and the use of mammalian cells in the initial screening of drugs.

Tissue engineering is a science which has implications with respect to many areas of clinical and cosmetic surgery. More particularly, tissue engineering relates to the replacement and/or restoration and/or repair of damaged and/or diseased tissues to return the tissue and/or organ to a functional state. For example, tissue engineering is useful in the provision of skin grafts to repair wounds occurring as a consequence of: contusions, or burns, or failure of tissue to heal due to venous or diabetic ulcers. Tissue engineering requires in vitro culturing of replacement tissue followed by surgical application of the tissue to a wound to be repaired.

The production of recombinant protein in cell expression systems is based either on prokaryotic cell expression or eukaryotic cell expression. The latter is preferred when post-translation modifications to the protein are required. Eukaryotic systems include the use of mammalian cells, e.g. Chinese Hamster Ovary cells; insect cells e.g. Spodoptera spp; or yeast e.g. Saccharomyces spp, Pichia spp. The large scale production of recombinant proteins requires a high standard of quality control since many of these proteins are used as pharmaceuticals, for example: growth hormone; leptin; erythropoietin; prolactin; TNF, interleukins; granulocyte colony stimulating factor (G-CSF); granulocyte macrophage colony stimulating factor (GM-C SF); ciliary neurotrophic factor (CNTF); cardiotrophin-1 (CT-1); leukemia inhibitory factor (LIF); oncostatin M (OSM); interferon, IFNα, IFNγ. Moreover, the development of vaccines, particularly subunit vaccines, (vaccines based on a defined antigen, for example gp120 of HIV), requires the production of large amounts of pure protein free from contaminating antigens which may provoke anaphylaxis. In some situations it is desirable to manufacture recombinant protein in cells that are differentiated and able to process the expressed polypeptide. Post-translation processing includes the proteolytic processing of precursor proteins and the addition or removal of chemical groups (e.g. phosphorylation, prenylation, glucosylation, farnesylation).

Moreover, mammalian cells are used in initial drug screening to determine whether a lead therapeutic (e.g. a small molecule agonist or antagonist, a monoclonal antibody, peptide therapeutic, nucleic acid aptamer, small inhibitory RNA (siRNA)) has efficacy before animal trials are undertaken.

There is a need to provide improved cell culture systems in which mammalian cells can be cultured to provide a population of cells that are as far as technically possible close to their natural state to enable the analysis of cell proliferation, differentiation and function in a reliable manner.

Cell culture systems are known in the art and have been available to the skilled person for many years. Cell culture typically involves the growth of cells in monolayer culture under sterile conditions in closed cell culture vessels. More recently cell culture systems have been developed that provide means by which cells can be cultured in 3 dimensions to more closely resemble the situation found in vivo. For example, WO2003/014334 discloses an in vitro cell culture method which provides a culture regime that allows prostate epithelial cells to form prostate-like-acini which closely resemble prostate acini found in vivo. These have utility in testing the efficacy of anti cancer agents with respect to controlling proliferation or metastasis of prostate cancer cells since transformed prostate epithelial cells also form acini in the cell culture system.

Furthermore, cell culture substrates are described in WO00/34454, the content of which is incorporated by reference in its entirety, which comprises microcellular polymeric materials which are described as polyHIPE polymers. These polymers form reticulate structures of pores that interconnect with one another to provide a substrate to which cells can attach and proliferate. The process for the formation of polyHIPEs allows pore volume to be accurately controlled with pore volume varying from 75% to 97%. Pore sizes can vary between 0.1 to 1000 micron and the diameter of the interconnecting members from a few microns to 100 microns. Furthermore the polyHIPEs can be combined with additional components that facilitate cell proliferation and/or differentiation. PolyHIPEs are therefore versatile substrates on which cells can attach and proliferate in a cell culture system. Processes for the preparation of polyHIPEs are well known in the art and also disclosed in WO2004/005355 and WO2004/004880 each of which is incorporated by reference in its entirety.

PolyHIPEs are commercially available and comprise for example oil phase monomers styrene, divinyl benzene and a surfactant, for example Span 80 sorbitan monooleate. In addition, the rigidity of the polymer formed during processing of the polyHIPE may be affected by the inclusion of a monomer such as 2-ethylhexyl acrylate. The process for the formation of polyHIPE from an emulsion is initiated by the addition of a catalyst such as ammonium persulphate.

The processes for the manufacture of polyHIPEs in WO00/34454, WO2004/005355 and WO2004/004880 describe various conditions for the formation polymers. For example, styrene concentration can vary from 15% (w/w) to 78% (w/w); surfactant concentration varies between 14% (w/w) and 15% (w/w) and the addition of the monomer 2-ethylhexyl acrylate varies between 60% (w/w) and 62% (w/w). Moreover, the disclosures in these applications relate to the production of unitary cell supports to which cells attach and grow. The resultant polyHIPEs formed by these processes have pore volumes that vary from 75% to 97%.

We herein describe a process for the formation of a polyHIPE that has superior properties specifically designed for the routine culture of cells, typically mammalian cells, when compared to polyHIPEs formed by prior art processes. The polyHIPEs thus formed have a porosity of around 90% and are further processed into thin membranes or layers (for example, by microtome sectioning) to produce a cell culture substrate comprising a plurality of thin polyHIPE adapted to fit existing cell culture vessels. The polyHIPE is also modified by the inclusion of organic monomers and polymers to provide a cell culture substrate tailored to specific cell-types. The cell culture system herein disclosed can be applied to both eukaryotic cells and prokaryotic cells to provide the means to produce cell cultures that mirror more closely in vivo conditions to provide a more reliable cell culture system that has applications, for example in tissue engineering, recombinant protein production and drug screening.

According to an aspect of the invention there is provided cell culture substrate comprising a plurality of sectioned microcellular polymeric material wherein the pore volume of the microcellular polymeric material is between 88% and 92%.

Pore volume is defined as the fraction of the total volume of the material that is comprised of pores, and is determined by the droplet fraction of the parent emulsion.

In a preferred embodiment of the invention said pore volume is about 90%.

We have determined that membranes of microcellular polymeric material with a pore volume of about 90% are a surprisingly effective substrate for cell growth. We have demonstrated that cell adherence, proliferation and function are significantly affected by the structure of the polymeric material. The cells adhere better to 90% porosity materials and proliferate well and show enhanced function over cells grown on polymeric materials with different porosities (for example, 95% pore volume). Furthermore, we have demonstrated that the proliferation and function of cells grown on 90% polymeric materials is significantly improved compared to the growth of cells on conventional 2-dimensional tissue culture plastic.

In a further preferred embodiment of the invention said substrate comprises a hydrophobic elastomer at a concentration of between 20% (w/w) and 40% (w/w) of the total monomer content.

In a preferred embodiment of the invention said hydrophobic elastomer is provided at a concentration of between 25% (w/w) and 35% (w/w). Preferably said concentration is selected from the group consisting of 26% (w/w); 27% (w/w); 28% (w/w); 29% (w/w); 30% (w/w); 31% (w/w); 32% (w/w); 33% (w/w); or 34% (w/w).

In a preferred embodiment of the invention said hydrophobic elastomer is provided at a concentration of 30% (w/w).

In a preferred embodiment of the invention said elastomer is selected from the group consisting of: 2-ethylhexyl acrylate; n-butyl acrylate and n-hexyl acrylate.

In a preferred embodiment of the invention said elastomer is 2-ethylhexyl acrylate. Preferably said 2-ethylhexyl acrylate is provided at between 28% (w/w) and 32% (w/w); preferably 2-ethylhexyl acrylate is provided at about 30% (w/w).

In a preferred embodiment of the invention said cell culture substrate comprises polyvinyl. Preferably said polyvinyl is polystyrene; preferably a polystyrene comprising a styrene monomer and divinylbenzene.

In a preferred embodiment of the invention said cell culture substrate comprises a surfactant.

In a preferred embodiment of the invention said surfactant is provided at a concentration of 20-30% (w/w) of the monomer phase of the emulsion; preferably 24-26% (w/w) and most preferably around 25% (w/w).

In a preferred embodiment of the invention said cell culture substrate comprises a plurality of sectioned microcellular polymeric material wherein said sections are 50-1000 microns thick; preferably said sections are approximately 500-750 microns thick. More preferably still said sections are 100-200 microns thick.

In a preferred embodiment of the invention said cell culture substrate comprises a plurality of sectioned microcellular polymeric material wherein said sections are 50-250 microns thick; preferably said sections are approximately 150 microns thick.

In an alternative preferred embodiment of the invention said cell culture substrate comprises a plurality of sectioned microcellular polymeric material wherein said sections are 50-150 microns thick; preferably said sections are approximately 120 microns thick.

In a preferred embodiment of the invention said sectioned microcellular material is approximately 300 microns thick.

In a preferred embodiment of the invention said cell culture substrate comprises a further organic monomer.

In a preferred embodiment of the invention said organic monomer is selected from the group consisting of: N-butyl methacrylate, n-hexyl methacrylate, cyclohexyl acrylate, cyclohexyl methacrylate, phenyl acrylate, phenyl methacrylate, 3-vinylbenzyl chloride, 4-vinylbenzyl chloride, para-acetoxystyrene.

In a yet further preferred embodiment of the invention said cell culture substrate comprises a further organic polymer.

In a preferred embodiment of the invention said organic polymer is selected from the group consisting of: Poly(n-butyl methacrylate), poly(n-hexyl methacrylate), poly(cyclohexyl acrylate), poly(cyclohexyl methacrylate), poly(phenyl acrylate), poly(phenyl methacrylate), poly(3-vinylbenzyl chloride), poly(4-vinylbenzyl chloride), poly(para-acetoxystyrene).

In a preferred embodiment of the invention said cell culture substrate comprises a surface that has been modified by the provision of a coating that facilitates the attachment, proliferation and/or differentiation of cells attached to the surface.

In a preferred embodiment of the invention said modification is the provision of a proteinaceous coating.

In a preferred embodiment of the invention said proteinaceous coating comprises at least one molecule selected from the group consisting of: laminin, collagen, for example cell supports like Matrigel, fibronectin, non-collagen based peptide matrices.

An example of such a non-collagen based peptide matrix is PuraMatrix™.

In an alternative preferred embodiment of the invention said proteinaceous coating comprises a poly-amino acid coating.

Poly-amino acids have properties that mimic proteins and in particular proteins to which cells can attach and grow. Poly-amino acids can be homopolymers or heteropolymers. Examples of poly amino acids useful in cell culture include poly L ornthine and poly L lysine. Proteinaceous coatings are well known in the art. For example see Culture of Animal Cells, Ian Freshney, Wiley-Liss 1994, which is incorporated by reference in its entirety.

In an alternative preferred embodiment of the invention the surface of said cell culture substrate is physically modified.

In a preferred embodiment of the invention said substrate comprises a surface that is modified by gas plasma treatment.

Gas plasma treatment of cell culture substrates is known in the art. The plasma treatment can be used to alter the physical properties of a cell culture surface. For example, ammonia and oxygen have been used as gas plasmas to improve cell attachment and proliferation on cell culture products. The process involves the excitation of gaseous products at low pressures and ambient temperatures by radio-frequency energy. The plasmas contain free electrons and other metastable particles which upon collision with polymeric surfaces can modify the surface by breaking chemical bonds. This creates free radicals which also modify the polymer surface.

According to a further aspect of the invention there is provided a cell culture vessel comprising a cell culture substrate according to the invention.

“Cell culture vessel” is defined as any means suitable to contain the above described cell culture substrate. Typically, an example of such a vessel is a petri dish; cell culture bottle or flask or multiwell culture dishes or well insert. Multiwell culture dishes are multiwell microtitre plates with formats such as 6, 12, 48, 96 and 384 wells which are typically used for compatibility with automated loading and robotic handling systems. Typically, high throughput screens use homogeneous mixtures of agents with an indicator compound that is either converted or modified resulting in the production of a signal. The signal is measured by suitable means (for example detection of fluorescence emission, optical density, or radioactivity) followed by integration of the signals from each well containing the cells, substrate/agent and indicator compound.

In a preferred embodiment of the invention said cell culture vessel comprising said cell culture substrate further comprises a cell and cell culture media.

In a preferred embodiment of the invention said cell is a eukaryotic cell; preferably said eukaryotic cell is selected from the group consisting of: a mammalian cell; a plant cell; a fungal cell; a slime mold.

In a preferred embodiment of the invention said mammalian cell is a primate cell; preferably said primate cell is a human cell.

In a preferred embodiment of the invention said mammalian cell is selected from the group consisting of: an epidermal keratinocyte; a fibroblast (e.g. dermal, corneal; intestinal mucosa, oral mucosa, bladder, urethral, prostate, liver) an epithelial cell (e.g. corneal, dermal, corneal; intestinal mucosa, oral mucosa, bladder, urethral, prostate, liver); a neuronal glial cell or neural cell; a hepatocyte or hepatocyte stellate cell; a mesenchymal cell; a muscle cell (cardiomyocyte, or myotube cell); a kidney cell; a blood cell (e.g. CD4+ lymphocyte, CD8+ lymphocyte; a pancreatic β cell; or an endothelial cell);

In a preferred embodiment of the invention said cell is a cell line derived from tumour tissue.

In an alternative preferred embodiment of the invention said mammalian cell is a stem cell.

In a preferred embodiment of the invention said stem cell is selected from the group consisting of: haemopoietic stem cell; neural stem cell; bone stem cell; muscle stem cell; mesenchymal stem cell; epithelial stem cell (derived from organs such as the skin, gastrointestinal mucosa, kidney, bladder, mammary glands, uterus, prostate and endocrine glands such as the pituitary); endodermal stem cell (derived from organs such as the liver, pancreas, lung and blood vessels); embryonic stem cell; embryonic germ cell; embryonal carcinoma stem cell.

In a preferred embodiment of the invention said embryonic stem cell/embryonic germ cell is a pluripotent cell and not a totipotent cell.

In an alternative preferred embodiment of the invention said cell is a prokaryotic cell; preferably a bacterial cell.

In a further preferred embodiment of the invention said cell or cell line is genetically modified.

In a preferred embodiment of the invention said cell culture vessel is a bioreactor; preferably said bioreactor is designed to scale-up the proliferation, differentiation and function of the said cell type.

According to an aspect of the invention there is provided a method for the culture of cells comprising the steps of:
i) providing a cell culture vessel comprising:
   a) cells;
   b) a cell culture substrate according to the invention;
   c) cell culture medium sufficient to support the growth of said cells; and
ii) providing cell culture conditions which promote the proliferation and/or differentiation and/or function of said cells.

In a preferred method of the invention said cells are mammalian cells; preferably human cells.

In a preferred method of the invention said cells are hepatocytes.

In an alternative preferred embodiment of the invention said cells are prokaryotic cells; preferably bacterial cells.

If microorganisms are used in the cell culture method according to the invention, they are grown or cultured in the manner with which the skilled worker is familiar, depending on the host organism. As a rule, microorganisms are grown in a liquid medium comprising a carbon source, usually in the form of sugars, a nitrogen source, usually in the form of organic nitrogen sources such as yeast extract or salts such as ammonium sulfate, trace elements such as salts of iron, manganese and magnesium and, if appropriate, vitamins, at temperatures of between 0° C. and 100° C., preferably between 10° C. and 60° C., while gassing in oxygen.

The pH of the liquid medium can either be kept constant, that is to say regulated during the culturing period, or not. The cultures can be grown batchwise, semi-batchwise or continuously. Nutrients can be provided at the beginning of the fermentation or fed in semi-continuously or continuously. The products produced can be isolated from the organisms as described above by processes known to the skilled worker, for example by extraction, distillation, crystallization, if appropriate precipitation with salt, and/or chromatography. To this end, the organisms can advantageously be disrupted beforehand. In this process, the pH value is advantageously kept between pH 4 and 12, preferably between pH 6 and 9, especially preferably between pH 7 and 8.

As described above, these media which can be employed in accordance with the invention usually comprise one or more carbon sources, nitrogen sources, inorganic salts, vitamins and/or trace elements.

Preferred carbon sources are sugars, such as mono-, di- or polysaccharides. Examples of carbon sources are glucose, fructose, mannose, galactose, ribose, sorbose, ribulose, lactose, maltose, sucrose, raffinose, starch or cellulose. Sugars can also be added to the media via complex compounds such as molasses or other by-products from sugar refining. The addition of mixtures of a variety of carbon sources may also be advantageous. Other possible carbon sources are oils and fats such as, for example, soya oil, sunflower oil, peanut oil and/or coconut fat, fatty acids such as, for example, palmitic acid, stearic acid and/or linoleic acid, alcohols and/or polyalcohols such as, for example, glycerol, methanol and/or ethanol, and/or organic acids such as, for example, acetic acid and/or lactic acid.

Nitrogen sources are usually organic or inorganic nitrogen compounds or materials comprising these compounds. Examples of nitrogen sources comprise ammonia in liquid or gaseous form or ammonium salts such as ammonium sulfate, ammonium chloride, ammonium phosphate, ammonium carbonate or ammonium nitrate, nitrates, urea, amino acids or complex nitrogen sources such as cornsteep liquor, soya meal, soya protein, yeast extract, meat extract and others. The nitrogen sources can be used individually or as a mixture.

Inorganic salt compounds which may be present in the media comprise the chloride, phosphorus and sulfate salts of calcium, magnesium, sodium, cobalt, molybdenum, potassium, manganese, zinc, copper and iron.

Inorganic sulfur-containing compounds such as, for example, sulfates, sulfites, dithionites, tetrathionates, thiosulfates, sulfides, or else organic sulfur compounds such as mercaptans and thiols may be used as sources of sulfur for the production of sulfur-containing fine chemicals, in particular of methionine.

Phosphoric acid, potassium dihydrogenphosphate or dipotassium hydrogenphosphate or the corresponding sodium-containing salts may be used as sources of phosphorus.

Chelating agents may be added to the medium in order to keep the metal ions in solution. Particularly suitable chelating agents comprise dihydroxyphenols such as catechol or protocatechuate and organic acids such as citric acid.

According to a further aspect of the invention there is provided a method to screen for an agent wherein said agent affects the proliferation, differentiation or function of a cell comprising the steps of:
i) providing a cell culture comprising at least one cell and a cell culture substrate according to the invention;
ii) adding at least one agent to be tested; and
iii) monitoring the activity of the agent with respect to the proliferation, differentiation or function of said cells.

In a preferred method of the invention said cell is a hepatocyte.

In a preferred method of the invention said screening method includes the steps of: collating the activity data in (iii) above; converting the collated data into a data analysable form; and optionally providing an output for the analysed data.

A number of methods are known which image and extract information concerning the spatial and temporal changes occurring in cells expressing, for example fluorescent proteins and other markers of gene expression, (see Taylor et al Am. Scientist 80: 322-335, 1992), which is incorporated by reference. Moreover, U.S. Pat. No. 5,989,835 and U.S. Ser. No. 09/031,271, both of which are incorporated by reference, disclose optical systems for determining the distribution or activity of fluorescent reporter molecules in cells for screening large numbers of agents for biological activity. The systems disclosed in the above patents also describe a computerised method for processing, storing and displaying the data generated.

The screening of large numbers of agents requires preparing arrays of cells for the handling of cells and the administration of agents. Assay devices, for example, include standard multiwell microtitre plates with formats such as 6, 12, 48, 96 and 384 wells which are typically used for compatibility with automated loading and robotic handling systems. Typically, high throughput screens use homogeneous mixtures of agents with an indicator compound which is either converted or modified resulting in the production of a signal. The signal is measured by suitable means (for example detection of fluorescence emission, optical density, or radioactivity) followed by integration of the signals from each well containing the cells, agent and indicator compound.

The term “agent” includes any small molecule, antibody, polypeptide, peptide, aptamer, double stranded or small inhibitory RNA. These can be an agonist or an antagonist.

Small molecule antagonists include chemotherapeutic agents useful in the treatment of diseases such as cancer.

Antibodies or immunoglobulins (Ig) are a class of structurally related proteins consisting of two pairs of polypeptide chains, one pair of light (L) (low molecular weight) chain (κ, or λ), and one pair of heavy (H) chains (γ, α, μ, δ and ε), all four linked together by disulphide bonds. Both H and L chains have regions that contribute to the binding of antigen and that are highly variable from one Ig molecule to another. In addition, H and L chains contain regions that are non-variable or constant. The L chains consist of two domains. The carboxy-terminal domain is essentially identical among L chains of a given type and is referred to as the “constant” (C) region. The amino terminal domain varies from L chain to L chain and contributes to the binding site of the antibody. Because of its variability, it is referred to as the “variable” (V) region. The variable region contains complementarity determining regions or CDR's which form an antigen binding pocket. The binding pockets comprise H and L variable regions which contribute to antigen recognition. It is possible to create single variable regions, so called single chain antibody variable region fragments (scFv's). If a hybridoma exists for a specific monoclonal antibody it is well within the knowledge of the skilled person to isolate scFv's from mRNA extracted from said hybridoma via RT PCR. Alternatively, phage display screening can be undertaken to identify clones expressing scFv's. Alternatively said fragments are “domain antibody fragments”. Domain antibodies are the smallest binding part of an antibody (approximately 13 kDa). Examples of this technology is disclosed in U.S. Pat. No. 6,248,516, U.S. Pat. No. 6,291,158, U.S. Pat. No. 6,127,197 and EP0368684 which are all incorporated by reference in their entirety.

Aptamers are small, usually stabilised, nucleic acid molecules which comprise a binding domain for a target molecule. A screening method to identify aptamers is described in U.S. Pat. No. 5,270,163 which is incorporated by reference. Aptamers are typically oligonucleotides which may be single stranded oligodeoxynucleotides, oligoribonucleotides, or modified oligodeoxynucleotide or oligoribonucleotides.

A more recent technique to specifically ablate gene function is through the introduction of double stranded RNA, also referred to as small inhibitory or interfering RNA (siRNA), into a cell which results in the destruction of mRNA complementary to the sequence included in the siRNA molecule. The siRNA molecule comprises two complementary strands of RNA (a sense strand and an antisense strand) annealed to each other to form a double stranded RNA molecule. The siRNA molecule is typically derived from exons of the gene which is to be ablated. The mechanism of RNA interference is being elucidated. Many organisms respond to the presence of double stranded RNA by activating a cascade that leads to the formation of siRNA. The presence of double stranded RNA activates a protein complex comprising RNase III which processes the double stranded RNA into smaller fragments (siRNAs, approximately 21-29 nucleotides in length) which become part of a ribonucleoprotein complex. The siRNA acts as a guide for the RNase complex to cleave mRNA complementary to the antisense strand of the siRNA thereby resulting in destruction of the mRNA. An agent based on a siRNA would have value in determining the function of a specific gene in cell proliferation and/or differentiation.

According to a further aspect of the invention there is provided a method for the identification of genes associated with cell differentiation comprising the steps of:
i) providing a cell culture comprising at least one cell and a cell culture substrate according to the invention;
ii) extracting nucleic acid from cells in said cell culture;
iii) contacting said extracted nucleic acid with a nucleic acid array; and
iv) detecting a signal which indicates the binding of said nucleic acid to a binding partner on said nucleic acid array.

In a preferred method of the invention said cell is a hepatocyte.

Preferably said method includes the additional steps of:
i) collating the signal(s) generated by the binding of said nucleic acid to said binding partner;
ii) converting the collated signal(s) into a data analysable form; and optionally;
iii) providing an output for the analysed data.

Methods used in the identification of cell differentiation markers and/or markers of cell transformation include immunogenic based techniques (e.g. using the cells as complex immunogens to develop antisera to for example cell surface markers and the like) nucleic acid based techniques (e.g. differential screening using cDNA from normal and transformed cells). Also, it has been known for many years that tumour cells produce a number of tumour cell specific antigens, some of which are presented at the tumour cell surface. These are generally referred to as tumour rejection antigens and are derived from larger polypeptides referred to as tumour rejection antigen precursors. Tumour rejection antigens are presented via HLA's to the immune system. The immune system recognises these molecules as foreign and naturally selects and destroys cells expressing these antigens. If a transformed cell escapes detection and becomes established a tumour develops. Vaccines have been developed based on dominant tumour rejection antigens to provide individuals with a preformed defense to the establishment of a tumour. The method according to the invention provides a means to identify tumour rejection antigens and precursors which will have utility with respect to the vaccine development to provoke the patients own immune system to deter the establishment of tumours.

According to a yet further aspect of the invention there is provided an in vitro method to analyse the development of cancerous cells from normal cells comprising
i) forming a preparation comprising a cell culture substrate according to the invention including cells;
ii) adding at least one agent capable of inducing cell transformation; and
iii) monitoring the effect, or not, of said agent on the transformation of said cells.

In a preferred method of the invention said cells are hepatocytes.

It is well known in the art that there are agents capable of transforming a normal cell into a transformed cell with many of the features of cancerous cells. These include, by example only, viruses, DNA intercalating agents, oncogenes and telomerase genes.

As used herein, the term “cancer” or “cancerous” refers to cells having the capacity for autonomous growth, i.e., an abnormal state or condition characterized by rapidly proliferating cell growth. The term is meant to include all types of cancerous growths or oncogenic processes, metastatic tissues or malignantly transformed cells, tissues, or organs, irrespective of histopathologic type or stage of invasiveness. The term “cancer” includes malignancies of the various organ systems, such as those affecting, for example, lung, breast, thyroid, lymphoid, gastrointestinal, and genito-urinary tract, as well as adenocarcinomas which include malignancies such as most colon cancers, renal-cell carcinoma, prostate cancer and/or testicular tumours, non-small cell carcinoma of the lung, cancer of the small intestine and cancer of the esophagus. The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. Exemplary carcinomas include those forming from tissue of the cervix, lung, prostate, breast, head and neck, colon and ovary. The term “carcinoma” also includes carcinosarcomas, e.g., which include malignant tumours composed of carcinomatous and sarcomatous tissues. An “adenocarcinoma” refers to a carcinoma derived from glandular tissue or in which the tumor cells form recognizable glandular structures. The term “sarcoma” is art recognized and refers to malignant tumours of mesenchymal derivation.

According to a further aspect of the invention there is provided a process for the formation of a microcellular polymeric material comprising the steps of:
i) forming a preparation comprising an high internal phase emulsion comprising a hydrophobic elastomer at a concentration of between 20% (w/w) and 40% (w/w);
ii) forming a preparation comprising a catalyst;
iii) combining the preparations in (i) and (ii); and
iv) incubating the combined preparation to allow formation of a high internal phase emulsion polymer.

In a preferred method of the invention said hydrophobic elastomer is provided at a concentration of between 25% (w/w) and 35% (w/w); preferably said hydrophobic elastomer is provided at a concentration of about 30% (w/w).

In a preferred method of the invention said elastomer is selected from the group consisting of: 2-ethylhexyl acrylate; n-butyl acrylate and n-hexyl acrylate.

In a preferred method of the invention the temperature of the preparation in ii) is heated to a temperature of between 50° C. and 80° C.

In a further preferred method of the invention said preparation in ii) is heated to 50° C. or 60° C. or 80° C.

In a further preferred method of the invention said preparation in i) comprises a styrene monomer.

In a further preferred method of the invention said preparation in i) comprises divinyl benzene.

In a yet further preferred method of the invention said preparation in i) comprises a surfactant that is provided at a concentration of 20-30% (w/w); preferably 24-26% (w/w) and most preferably around 25% (w/w).

In a preferred method of the invention the preparation in i) comprises 60% (w/w) styrene; 30% (w/w) 2-ethylhexyl acrylate; 10% (w/w) divinylbenzene and 25% surfactant.

In a preferred method of the invention said high internal phase emulsion polymer in step iv) is sectioned; preferably said polymer is sectioned into a thin membrane or layer.

In a preferred method of the invention said polymer is engineered into a thin membrane or layer of approximately 50-150 microns thick; preferably said membranes are approximately 120 microns thick.

According to a further aspect of the invention there is provided a high internal phase emulsion polymer obtained or obtainable by the process according to the invention.

In a preferred embodiment of the invention said a high internal phase emulsion polymer has a pore volume of about 90%.

According to a further aspect of the invention said high internal phase emulsion polymer is for use in the culture of cells.

In a preferred embodiment of the invention the high internal phase emulsion polymer has a pore volume of around 90%; preferably 90%.

According to a further aspect of the invention there is provided the use of a substrate comprising a high internal phase emulsion polymer to determine the liver toxicity of an agent.

In a preferred embodiment of the invention said agent is a chemotherapeutic agent. In an alternative preferred embodiment of the invention said agent is a viral gene therapy vector.

According to a further aspect of the invention there is provided a method to test the liver toxicity of an agent comprising the steps of:
i) providing a cell culture comprising at least one hepatocyte cell and a cell culture substrate according to any of claims 1-31;
ii) adding at least one agent to be tested; and
iii) monitoring the activity of the agent with respect to the proliferation, differentiation or function of said hepatocyte cells as a measure of toxicity of the agent.

In a preferred method according to claim 83 wherein said agent is a chemotherapeutic agent.

In an alternative preferred method of the invention said agent is a viral gene therapy vector.

According to a further aspect of the invention there is provided a method for the growth and differentiation of a keratinocyte and/or keratinocyte precursor stem cell comprising:
i) forming a preparation comprising a cell culture substrate according to the invention, fibroblast feeder cells and cell culture medium;
ii) culturing said feeder cells to provide a cell culture substrate that is substantially coated with said feeder cells;
iii) contacting said coated substrate with keratinocytes and/or keratinocyte precursor stem cells; and
iv) culturing the combined cell preparation under conditions conducive to the growth and differentiation of said keratinocytes and/or keratinocyte precursor stem cells.

In a preferred method of the invention said fibroblast feeder cells are dermal fibroblasts.

In an alternative preferred method of the invention said fibroblast feeder cells are selected from the group consisting of: corneal fibroblasts, intestinal mucosa fibroblasts, oral mucosa fibroblasts, urethral fibroblasts, or bladder fibroblasts.

In a further preferred method of the invention said keratinocytes are epidermal keratinocytes.

In a preferred method of the invention said fibroblasts are human fibroblasts.

In a further preferred method of the invention said keratinocytes are human keratinocytes.

In a preferred method of the invention said preparation further comprises collagen.

In a preferred method of the invention collagen is type 1 collagen.

In a further preferred method of the invention said collagen is provided as a gel.

In an alternative preferred method of the invention said collagen is provided in a solution.

In a further preferred method of the invention at least said keratinocytes are displaced to contact air thereby inducing keratinocyte stratification.

In a preferred method of the invention there is provided a method to test an agent comprising:
i) forming a preparation according to the invention which includes an agent to be tested;
ii) monitoring the effect of said agent on keratinocyte cell growth and/or differentiation when compared to a control preparation that does not include said agent.

According to a further aspect of the invention there is provided an apparatus for the culture of cells comprising a cell culture substrate according to any of claims 1-31, a cell culture vessel and an insert adapted to co-operate with said cell culture vessel and contain said cell culture substrate and said cells.

In a preferred embodiment of the invention said cell culture substrate comprises fibroblasts and keratinocytes.

According to a yet further aspect of the invention there is provided the use of a substrate according to the invention for the preparation of differentiated skin composite.

Throughout the description and claims of this specification, the words “comprise” and “contain” and variations of the words, for example “comprising” and “comprises”, means “including but not limited to”, and is not intended to (and does not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

An embodiment of the invention will now be described by example only and with reference to the following figures:

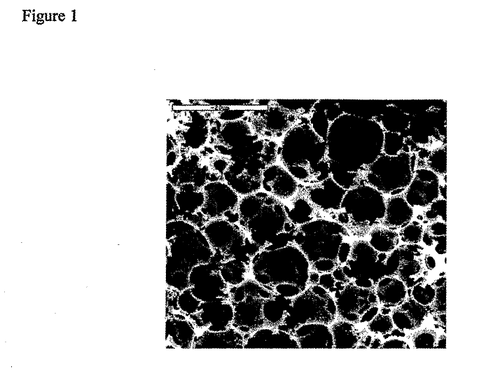

FIG. 1 is a scanning electron micrograph (SEM) image of a typical PolyHIPE material. The spherical cavities in FIG. 1 are voids, the holes joining adjacent voids are called interconnects. Scale bar=20□m;

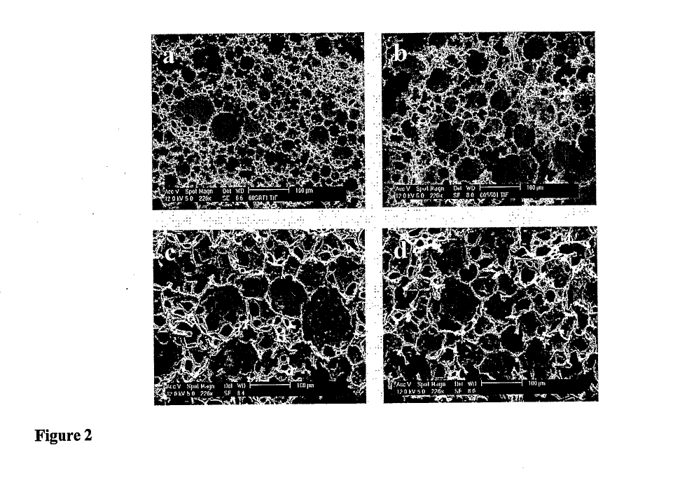

FIG. 2 shows SEM images of PolyHIPE materials prepared with different aqueous phase temperatures: (a) room temperature; (b) 50° C.; (c) 60° C.; (d) 80° C. Scale bar=100 □m;

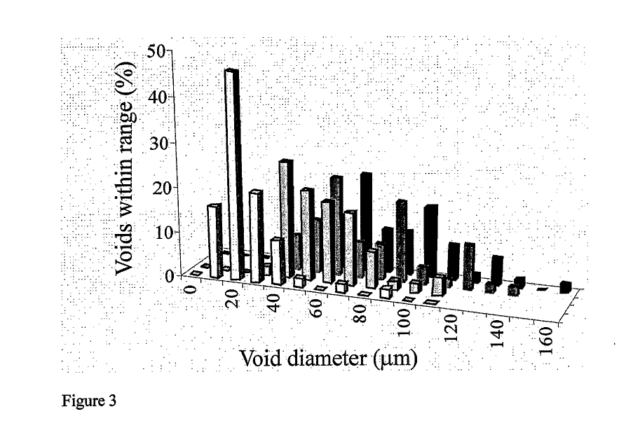

FIG. 3 illustrates the influence of aqueous phase temperature on void diameter distribution. From front to back: room temperature, 50° C., 60° C., 80° C.;

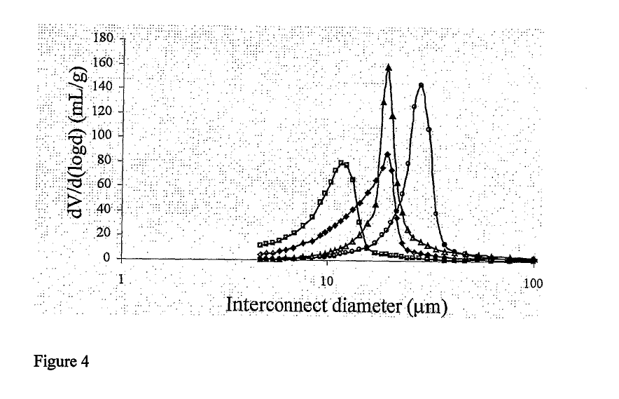

FIG. 4 illustrates interconnect size distribution of PolyHIPE materials produced using different aqueous phase temperatures: room temperature (□); 50° C. (⋄); 60° C. (Δ); 80° C. (◯);

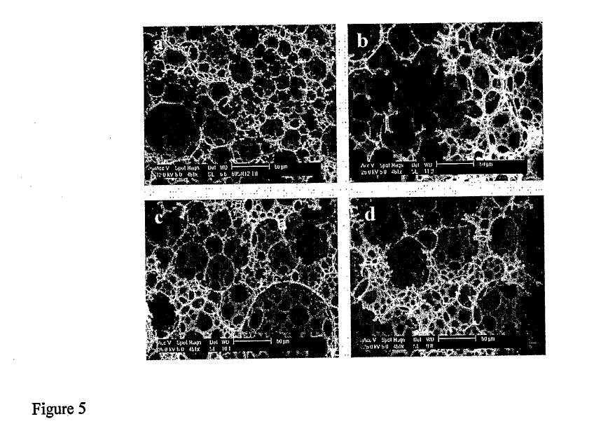

FIG. 5 shows the influence of aqueous phase additives on PolyHIPE morphology: (a) no additive; (b) 1.5% (w/v) PEG (Mₙ=300); (c) 4% (v/v) methanol; (d) 1.5% (v/v) THF. Scale bar=50 □m.

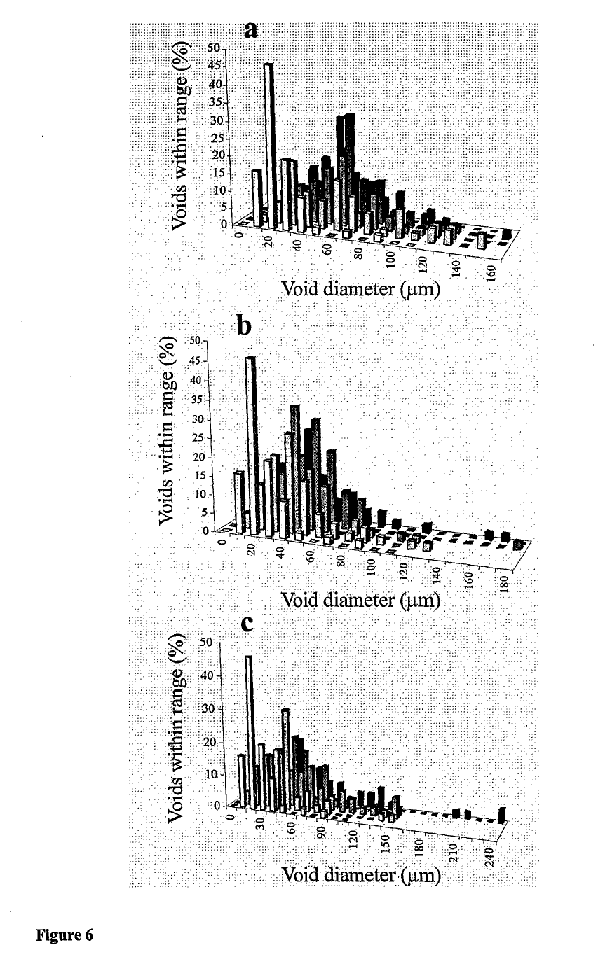

FIG. 6 illustrates void diameter distribution plots for PolyHIPE materials prepared with aqueous phase additives: (a) PEG (from front to back: no PEG, 0.2%, 0.4%, 0.8%, 1.5%); (b) methanol (from front to back: no methanol, 1%, 2%, 3%, 4%); (c) THF (from front to back: no THF, 0.4%, 0.8%, 1%, 1.5%). PEG Mₙ=300; all percentages expressed as v/v, except PEG which is w/v. In each case the aqueous phase was kept at room temperature during emulsion preparation;

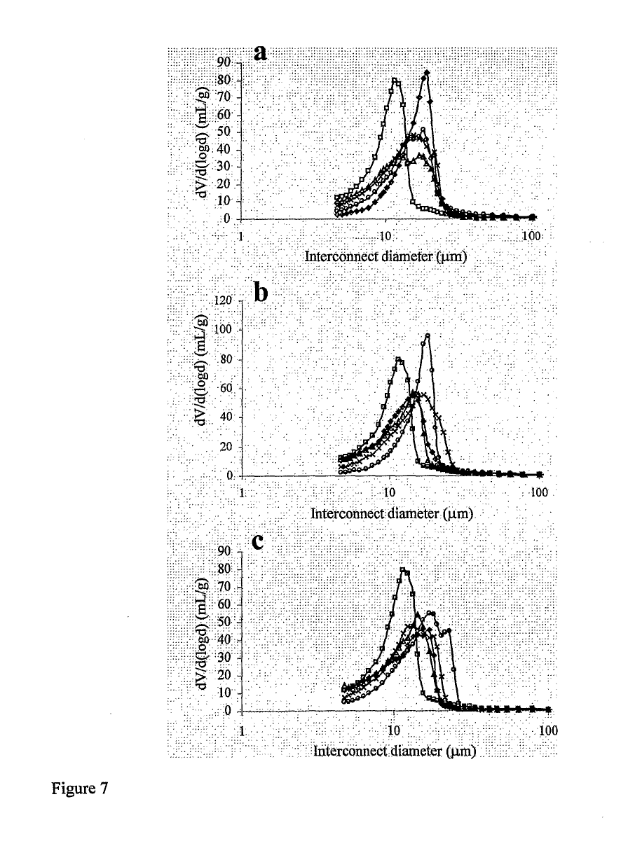

FIG. 7 illustrates interconnect size distribution of PolyHIPE materials produced using different aqueous phase additives: (a) PEG (□ no PEG, Δ 0.2%, x 0.4%, ◯ 0.8%, ⋄1.5%); (b) methanol (□ no methanol, Δ 1%, ⋄ 2%, ◯3%, x 4%); (c) THF (□ no THF, ⋄ 0.4%, Δ 0.8%, x 1%, ◯ 1.5%). PEG Mₙ=300; all percentages expressed as v/v, except PEG which is w/v. In each case the aqueous phase was kept at room temperature during emulsion preparation;

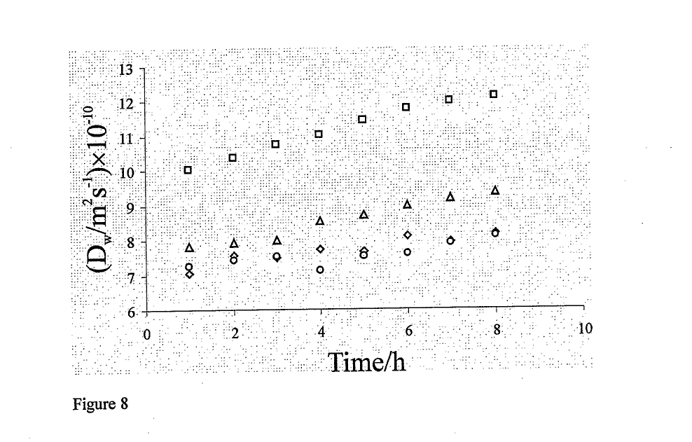

FIG. 8 illustrates self diffusion coefficient of water in HIPEs prepared with different aqueous phase additives (⋄ no additive; □ 1.5% THF; Δ 1.5% PEG; ◯ 2% methanol). PEG Mₙ=300; all percentages expressed as v/v, except PEG which is w/v. In each case the aqueous phase was kept at room temperature during emulsion preparation;

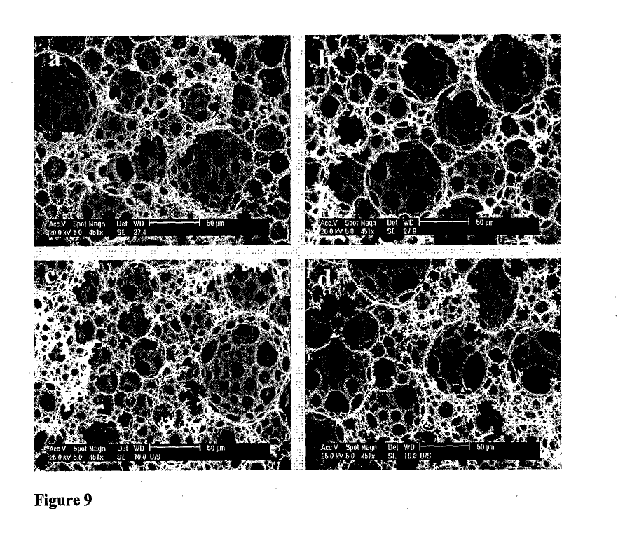

FIG. 9 shows SEM images of PolyHIPE materials prepared with different surfactant concentrations (Cₛ) in the presence of aqueous phase additives: 1.5% THF, C_{S}=20% (a); 1.5% THF, C_{S}=30% (b); 4% methanol, Cₛ=20% (c); 4% methanol, Cₛ=30% (d). Scale bar=50 □m. PEG Mₙ=300; all percentages expressed as v/v, except PEG which is w/v. In each case the aqueous phase was kept at room temperature during emulsion preparation.

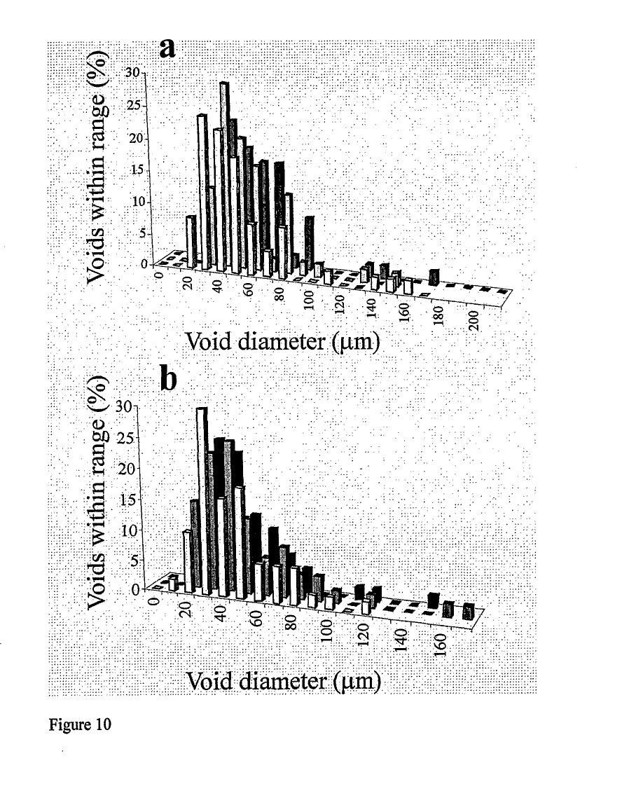

FIG. 10 illustrates void diameter distribution plots for PolyHIPE materials prepared with different surfactant concentrations in the presence of additives: 1.5% THF (a); 4% methanol (b). From front to back: Cₛ=30, 25 and 20% (w/w). PEG Mₙ=300; all percentages expressed as v/v, except PEG which is w/v. In each case the aqueous phase was kept at room temperature during emulsion preparation.

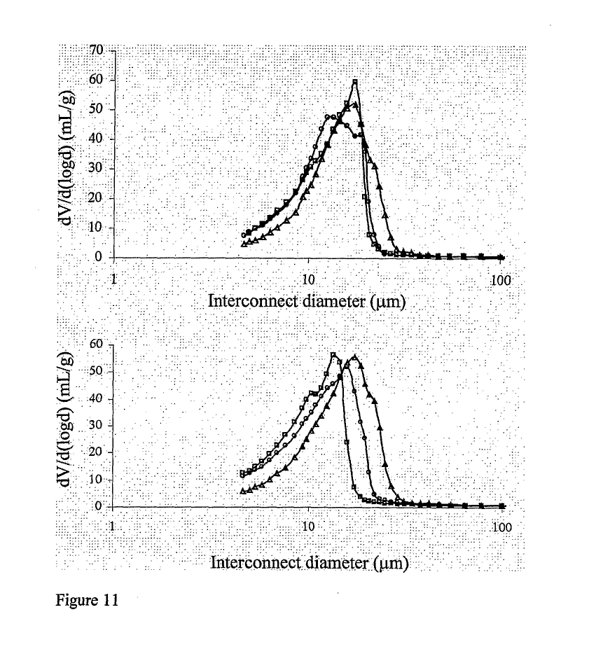

FIG. 11 illustrates interconnect size distribution of PolyHIPE materials produced using different surfactant concentrations (Cₛ) in the presence of aqueous phase additives: (a) 1.5 vol. % THF; (b) 4 vol. % methanol (□: Cₛ=20%; Δ: Cₛ=25%; ◯: Cₛ=30%; all percentages expressed as v/v). In each case the aqueous phase was kept at room temperature during emulsion preparation.

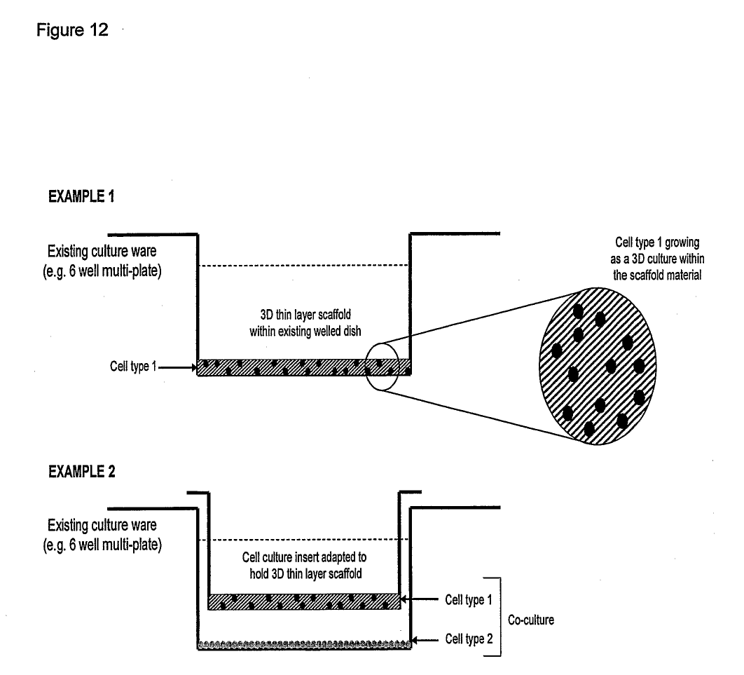

FIG. 12 illustrates an example application of styrene-based polyHIPE scaffolds as thin membranes adapted for use in existing cell culture vessels such as a multi-welled plate or well insert.

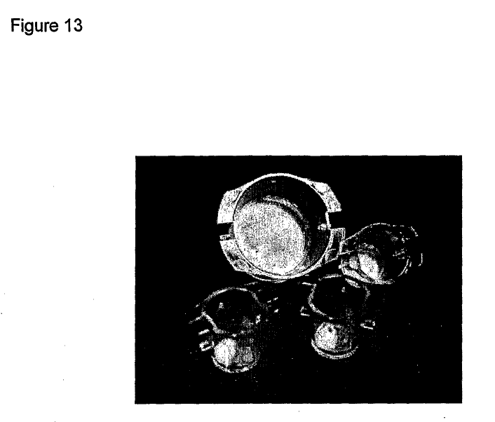

FIG. 13 shows a photograph of prototype well inserts carrying the 90% pore volume polystyrene scaffold at 120 microns thick. These examples are of inserts designed to fit into 6-welled (large insert) and 12-welled (small inserts) culture plates.

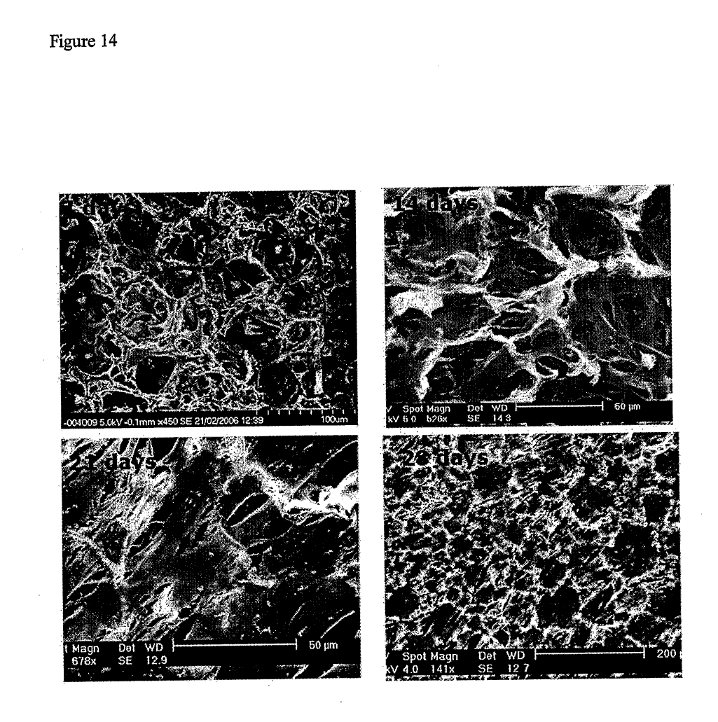

FIG. 14 is a SEM showing MG63 osteoblasts cultured on 90% pore volume polystyrene scaffolds for 7-28 days in vitro. These materials have been adapted for use in existing cell culture plastic-ware as illustrated in FIG. 12.

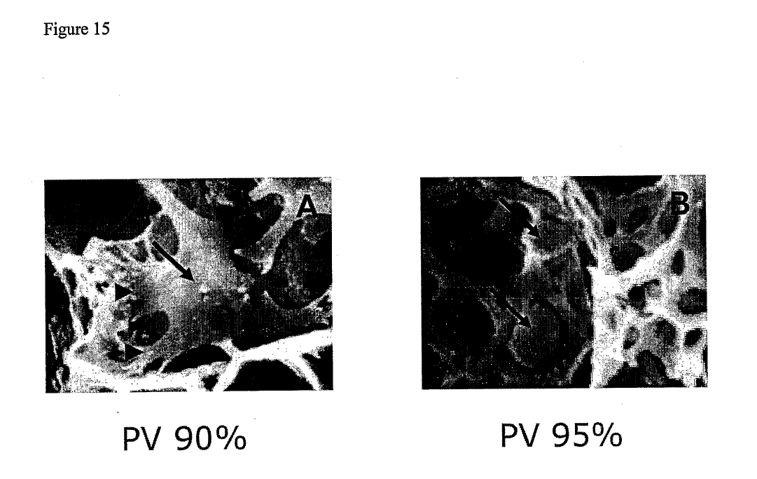

FIG. 15 demonstrates that the preparation and structural characteristics of the polymer affect the growth of cells within the scaffold (example: 90% versus 95% pore volume). This example shows how cell morphology is affected. Scanning electron micrographs of MG63 osteoblasts cultured on polystyrene scaffolds for 7 days in vitro. These materials were produced using pore volumes (PV) of 90% and 95%. (A) Osteoblasts (arrow) grown on 90% polymers spread out and exhibited numerous lamellipodia (arrowheads) enhancing interactions with neighbouring cells. (B) However, cells (arrows) grown 95% polymers maintained a rounded appearance and produced fewer if any lamellipodia. (Images are of similar magnification).

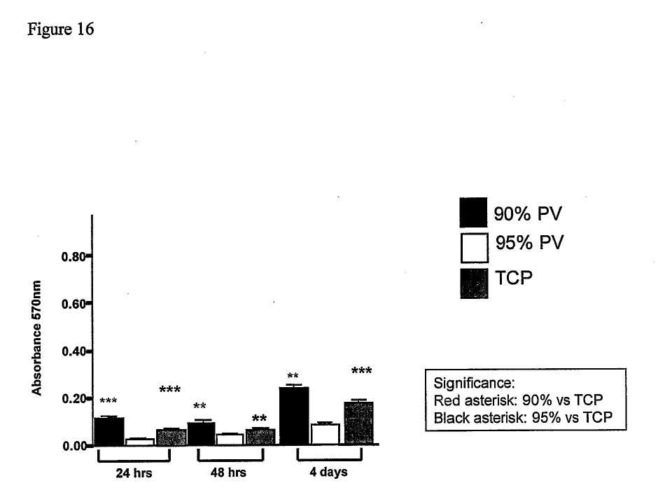

FIG. 16 illustrates how the structure of the growth substrate can influence cell behaviour. The data show significant differences in the proliferation rate of cells grown on various types of substrate. Specifically note the comparison between polymers of 90% and 95% pore volumes. This demonstrates the importance of tailoring these scaffolds for cell growth. The figure shows data from a MTT cell proliferation assay of cultured MG63 osteoblasts grown on either 90% or 95% pore volume (PV) polystyrene scaffolds, or flat, conventional tissue culture plastic (TCP). Cells were seeded at 1×10⁶ cells per well. Bars represent the mean±SEM, n=3. Note that cell proliferation is significantly greater on 90% scaffolds compared to TCP and 95% PV materials. These data also show that cells proliferate the least on scaffolds made with 95% PV.

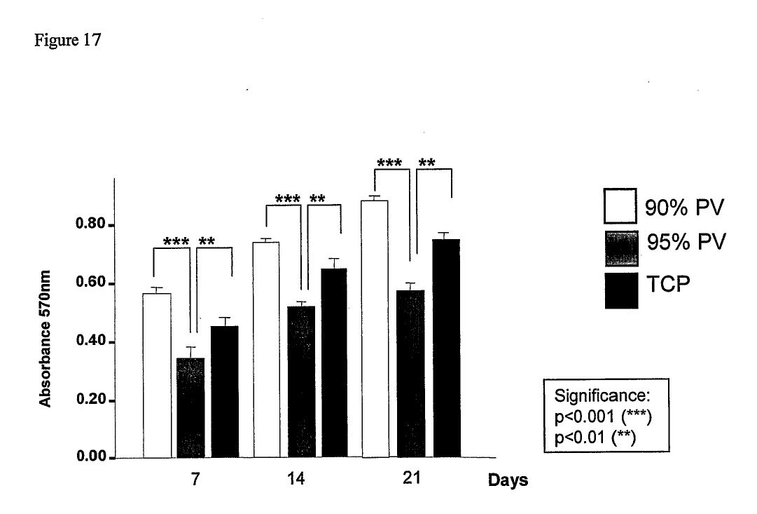

FIG. 17 illustrates how the structure of the growth substrate can influence cell behaviour. The data show significant differences in the proliferation rate of cells grown on various types of substrate. Specifically note the comparison between polymers of 90% and 95% pore volumes. This demonstrates the importance of tailoring these scaffolds for cell growth. The figure shows data from a MTT cell proliferation assay of cultured bone marrow derived mesenchymal stem cells (MSCs) grown on either 90% or 95% pore volume (PV) polystyrene scaffolds, or flat conventional tissue culture plastic (TCP). Cells were seeded at 1×10⁶ cells per well. Bars represent the mean±SEM, n=3. Again, these data show that cell proliferation is significantly greater on 90% scaffolds compared to TCP and 95% PV materials. In addition, cells proliferate the least on scaffolds made with 95% PV.

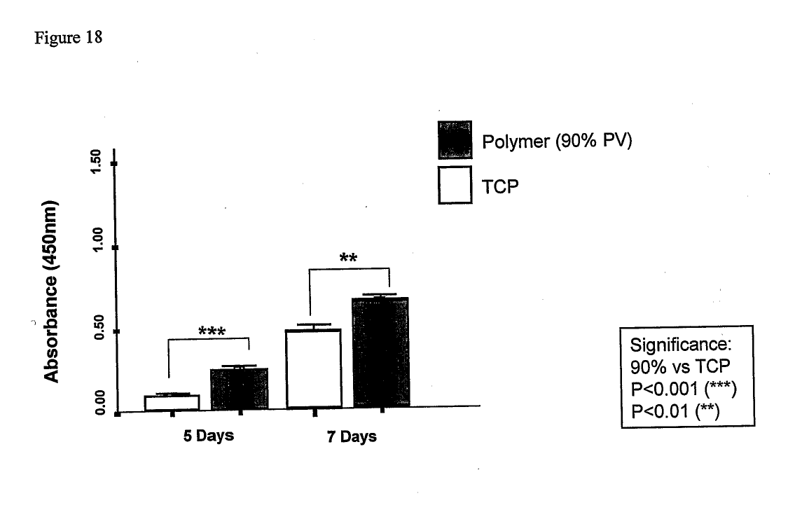

FIG. 18 shows significant differences in the function of cells grown on 3-dimensional 90% pore volume polystyrene scaffolds compared to their growth on 2-dimensional conventional tissue culture plastic. Assay measuring the levels of alkaline phosphatase in MG63 osteoblasts cultured on 90% pore volume (PV) scaffolds compared to flat, conventional tissue culture plastic (TCP) for 5 and 7 days. Cells were seeded at 1×10⁶ cells per well. Values have been normalized to account for any differences in cell number. Bars represent the mean±SEM, n=3. Note that alkaline phosphatase levels are significantly higher in cultures of osteoblasts grown on 3-dimensional polystyrene compared to flat polystyrene surfaces. These data show enhanced activity of these cells when grown on the 3-dimensional scaffold compared to conventional 2-dimensional culture plastic.

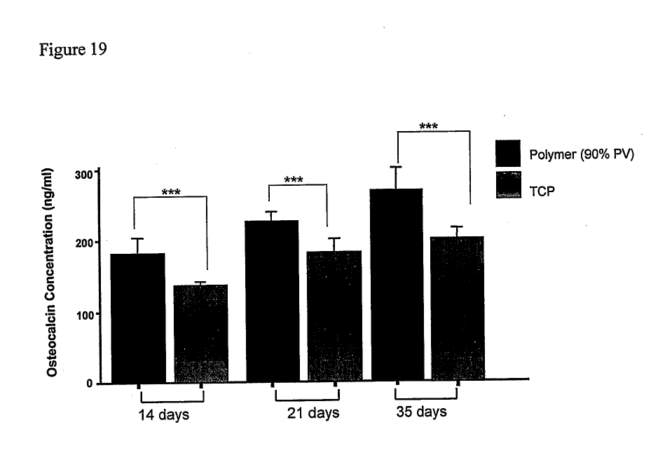

FIG. 19 shows significant differences in the function of cells grown on 3-dimensional 90% pore volume polystyrene scaffolds compared to their growth on 2-dimensional conventional tissue culture plastic. Assay measuring the levels of osteocalcin in bone marrow derived MSCs induced to form bone nodules in response to dexamethasone. Cells were cultured on either 90% pore volume (PV) polystyrene scaffolds or flat, conventional tissue culture plastic (TCP) for 14 to 35 days. Cells were seeded at 1×10⁶ cells per well. Values have been normalized to account far any differences in cell number. Bars represent the mean±SEM, n=3. Note that osteocalcin concentrations are significantly higher in cultures of differentiating cells grown on 3-dimensional polystyrene compared to flat polystyrene surfaces. These data again show enhanced activity of these cells when grown on the 3-dimensional scaffold compared to conventional 2-dimensional culture plastic.

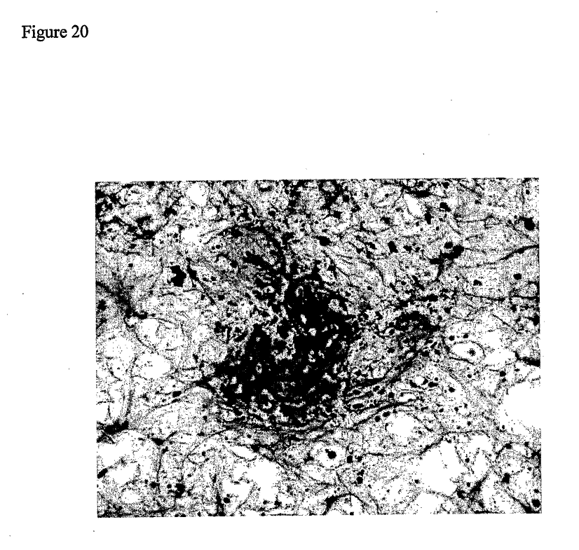

FIG. 20 is a photomicrograph of Von Kossa staining showing the formation of a centrally located bone nodule. The bone nodule was derived from mesenchymal stems induced to differentiate with dexamethasone when grown within a 90% pore volume polystyrene scaffold. Cells are counterstained with Mayor's Haematoxylin.

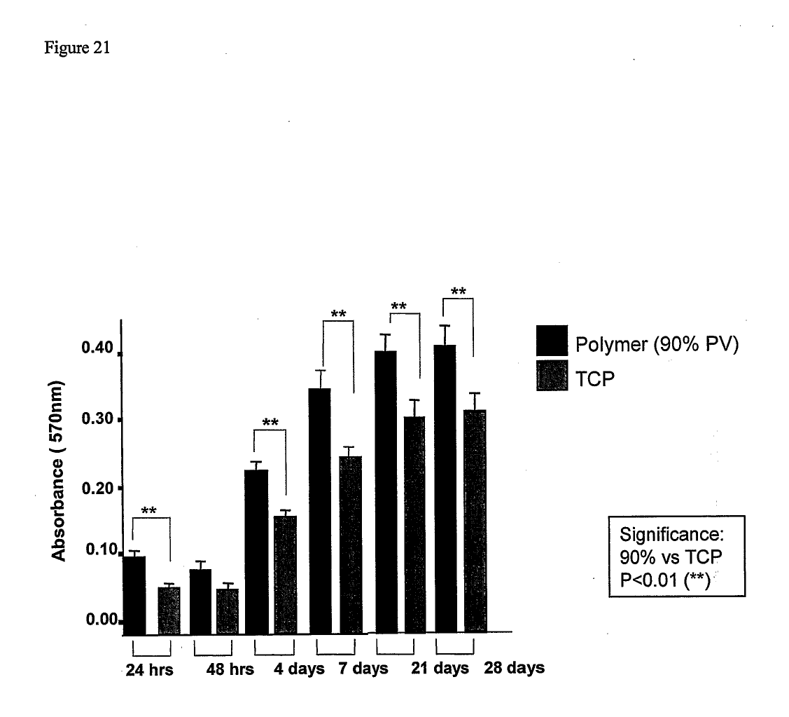

FIG. 21 illustrates how the structure of the growth substrate can influence cell behaviour. The data exemplify the advantage of growing cells within a 90% pore volume polystyrene scaffold compared to conventional tissue culture plastic. The figure shows data from a MTT cell proliferation assay of cultured HEP G2 hepatocytes grown on either 90% pore volume (PV) polystyrene scaffold or flat, conventional tissue culture plastic (TCP). Cells were seeded at 1×10⁶ cells/well. Bars represent the mean±SEM, n=3. Note that cell proliferation is significantly greater on 90% scaffolds compared to 2-dimensional TCP.

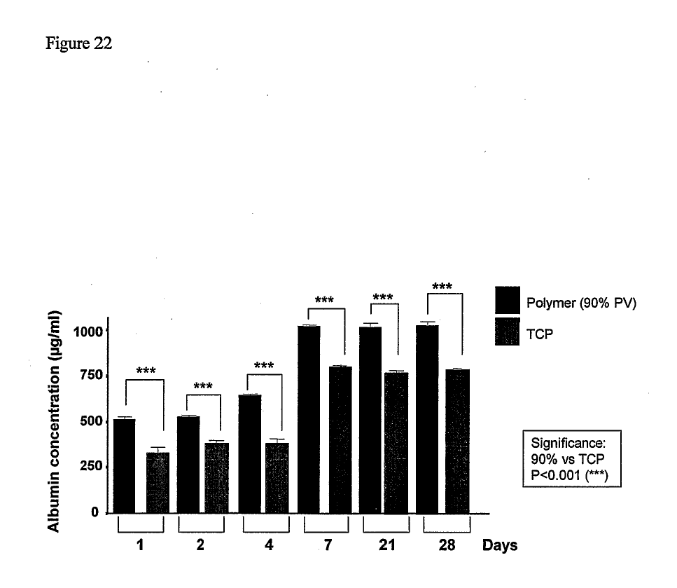

FIG. 22 illustrates how the structure of the growth substrate can influence cell function. The data exemplify the advantage of growing cells within a 90% pore volume polystyrene scaffold compared to conventional tissue culture plastic. Assay measuring the levels of albumin production from HEP G2 hepatocytes cultured on either 90% pore volume (PV) polystyrene scaffolds or flat, conventional tissue culture plastic (TCP) for 1 to 28 days. Cells were seeded at 1×10⁶ cells per well. Values have been normalized to account for any differences in cell number. Bars represent the mean±SEM, n=3. Note that albumin concentrations are significantly higher in cultures of differentiating cells grown on 3-dimensional polystyrene compared to flat polystyrene surfaces. These data again suggest enhanced activity of these cells when grown on the 3-dimensional scaffold compared to those cultured on the flat surface of conventional plastic-ware.

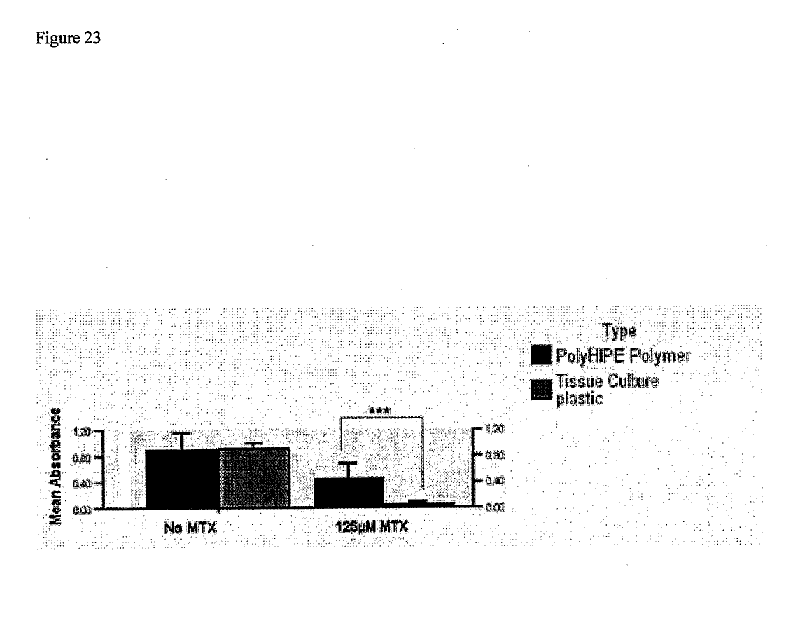

FIG. 23 illustrates how the structure of the growth substrate can influence cell function, in this case, the enhanced tolerance of cells to cytotoxic challenge. The data exemplify the advantage of growing cells within a 90% pore volume polystyrene scaffold compared to conventional tissue culture plastic. The figure shows data from a MTT cell proliferation assay of cultured HEP G2 hepatocytes grown on either 90% pore volume (PV) polystyrene scaffold or flat, conventional tissue culture plastic (TCP) for 3 days in the presence (125 microM) or absence of the cytotoxin methotrexate (DNA synthesis inhibitor). Cells were seeded at 1×10⁶ cells/well. Bars represent the mean±SEM, n=3. Note that cell proliferation is significantly greater on 90% scaffolds compared to 2-dimensional TCP. These data suggest that cells grown on scaffolds are more tolerant to this cytotoxin under these growth conditions.

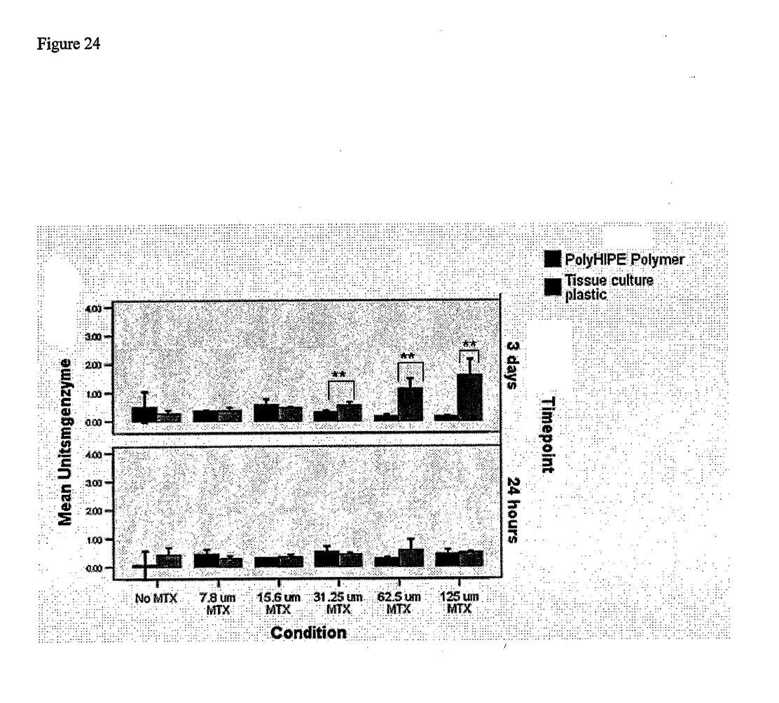

FIG. 24 illustrates how the structure of the growth substrate can influence cell function and further exemplify the differences in growing cells within a 90% pore volume polystyrene scaffold compared to conventional tissue culture plastic. Assay measuring the levels of transglutaminase in cultures of HEP G2 hepatocytes grown on either 90% pore volume (PV) polystyrene scaffolds or flat, conventional tissue culture plastic (TCP) for 1 to 3 days. Cells were seeded at 1×10⁶ cells per well. Values have been normalized to account for any differences in cell number. Bars represent the mean±SEM, n=3. Transglutaminase is a protein cross-linking enzyme known to be expressed by hepatocytes and is induced as hepatocytes enter apoptosis. Note that levels of transglutaminase are significantly higher in hepatocyte cultures grown on flat polystyrene surfaces compared to 3-dimensional polystyrene when challenged with increasing concentrations of the cytotoxin methotrexate. These data further suggest that cells on scaffolds are more tolerant to these levels of cytotoxic challenge which may be consequence of their growth under less stressful conditions unlike those experienced by cells grown as 2-dimensional monolayers;

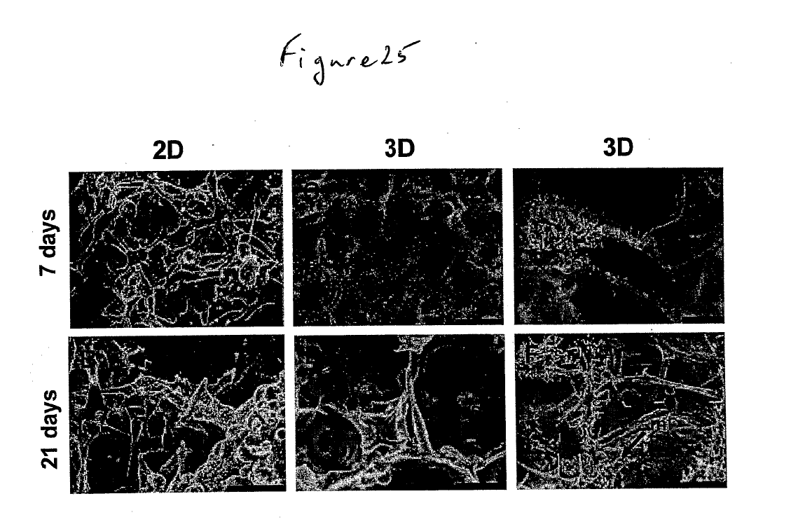

FIG. 25: Scanning electron micrographs showing HepG2 hepatocytes cultured on 2-D (A,B) and 3-D (C-F) polystyrene substrates for either 7 days (A,C,E) or 21 days (B,D,F). Hepatocytes grown on 2-D substrates appeared significantly more heterogeneous in structure (A,B), compared to cells grown on 3-D surfaces (C). A decreased seeding density enabled visualisation of individual cells grown on 3-D scaffolds (sc) (D). HepG2 cells developed complex 3-D shapes and interactions with neighbouring cells (D). Higher magnification images revealed the expression of large numbers of micro-villi (mv) on the surface of cells (E,F). There were consistently greater numbers of micro-villi on cells grown in 3-D (C-F) compared to cells grown on 2-D surfaces (A,B). Scale bars: A-D 25 μm; E,F 5 μm.

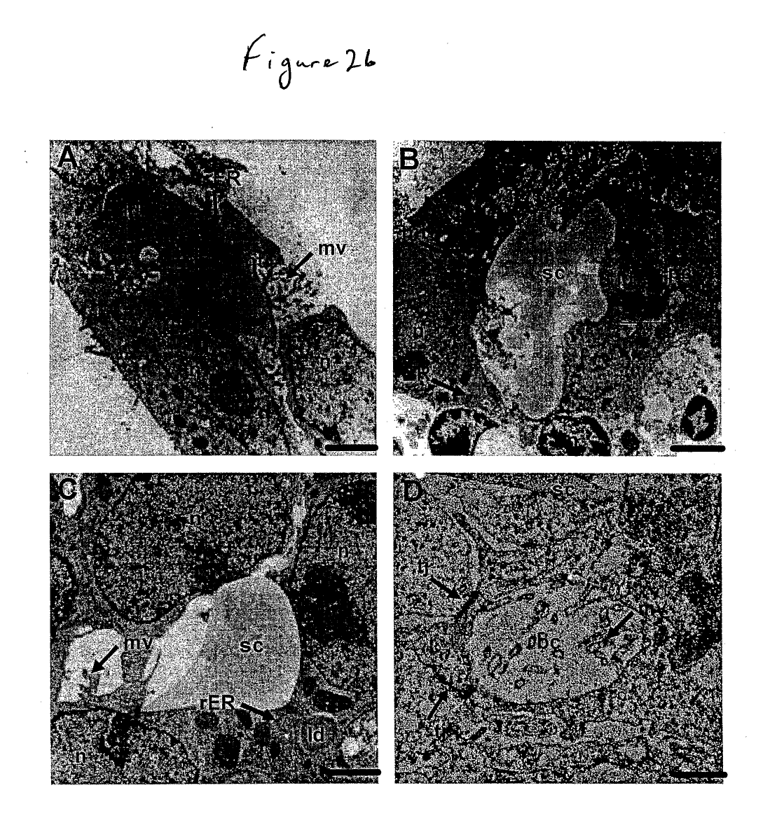

FIG. 26: Transmission electron micrographs showing the ultra-structural features of HepG2 cells cultured on either 2-D or 3-D surfaces for 21 days. (A) HepG2 cells cultured on 2-D plastic exhibited numerous clearly identifiable organelles, including nuclei (n), mitochondria (mt), rough endoplasmic reticulum (rER), micro-villi (mv), and lipid droplets (ld). (B,C) HepG2 cells cultured on polystyrene scaffolds (sc) grow in close association with the polymer, completely surrounding struts of the material as shown. Imaging showed that cells grown in 3-D also displayed an array of cellular organelles such as nuclei (n), mitochondria (mt), rough endoplasmic reticulum (rER), micro-villi (mv), lipid droplets (ld) and peroxisomal clusters (pc). (D) High magnification micrograph showing the formation of tight junction (tj) complexes between adjacent cells. The void formed in between cells closely resembles a bile canaliculus (bc) into which project micro-villi (mv). Scale bars: A,B 2 μm; C 1 μm; D 500 nm.

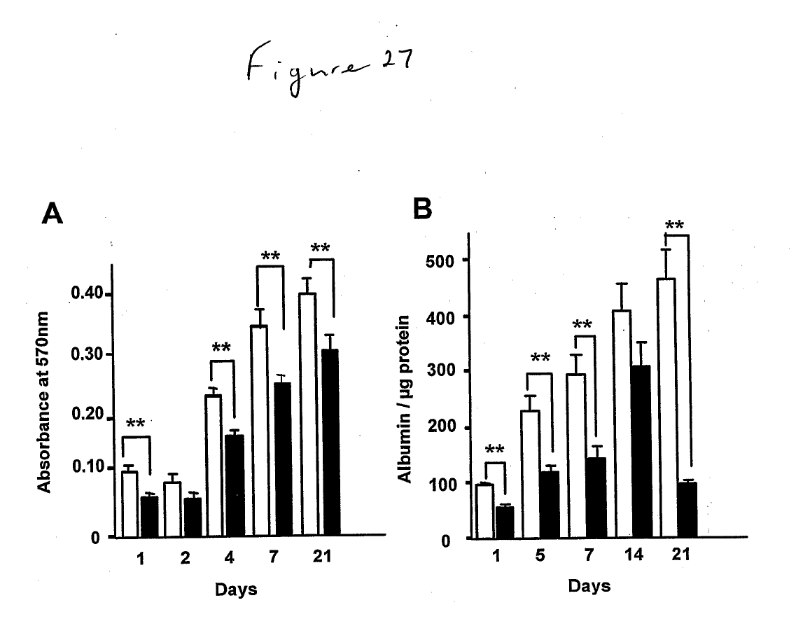

FIG. 27: Performance of HepG2 cells cultured on 2-D (solid bars) and 3-D (open bars) polystyrene substrates cultured for 21 days. (A) Assessment of cell viability using MTT assay. (B) Production of albumin secreted by HepG2 cells into the culture medium. Albumin secretion was normalized to the total amount of protein per well. For both experiments, cells were seeded at 1×10⁶ cells/well. Data represent the mean±SEM for three independent repeats. Significance is denoted by **p<0.01 using the Mann Whitney U test.

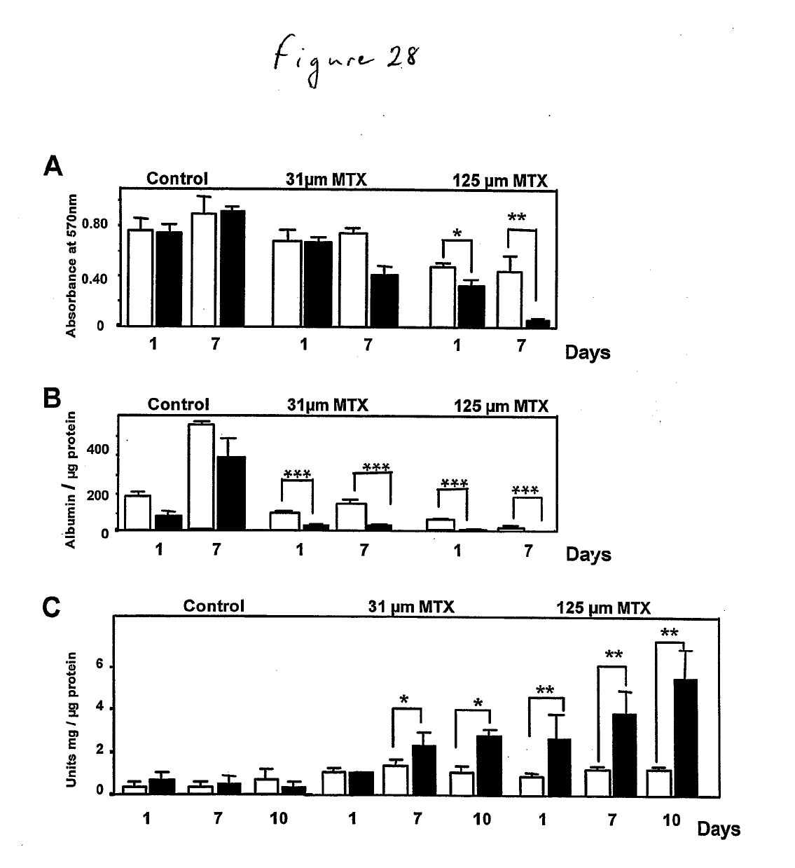

FIG. 28: Performance of HepG2 cells cultured on 2-D (solid bars) and 3-D (open bars) substrates when challenged by the cytotoxin, methotrexate (MTX). Data show cells were treated either with vehicle alone (control), or 31 μM MTX, or 125 μM MTX for up to 10 days. (A) Measure of cell viability using MTT assay. (B) Determination HepG2 cell metabolic activity by measurement of albumin secretion into the culture medium. Albumin levels were normalized to the total amount of protein per well. (C) Assessment of cell damage as determined by transglutaminase activity. Enzyme levels were normalized to the total amount of protein per well. For each experiment (A-C), cells were seeded at 1×10⁶ cells/well. Data represent the mean±SEM for three independent repeats. Significance is denoted by *p<0.05, **p<0.01 and ***p<0.001 using the Mann Whitney U test.

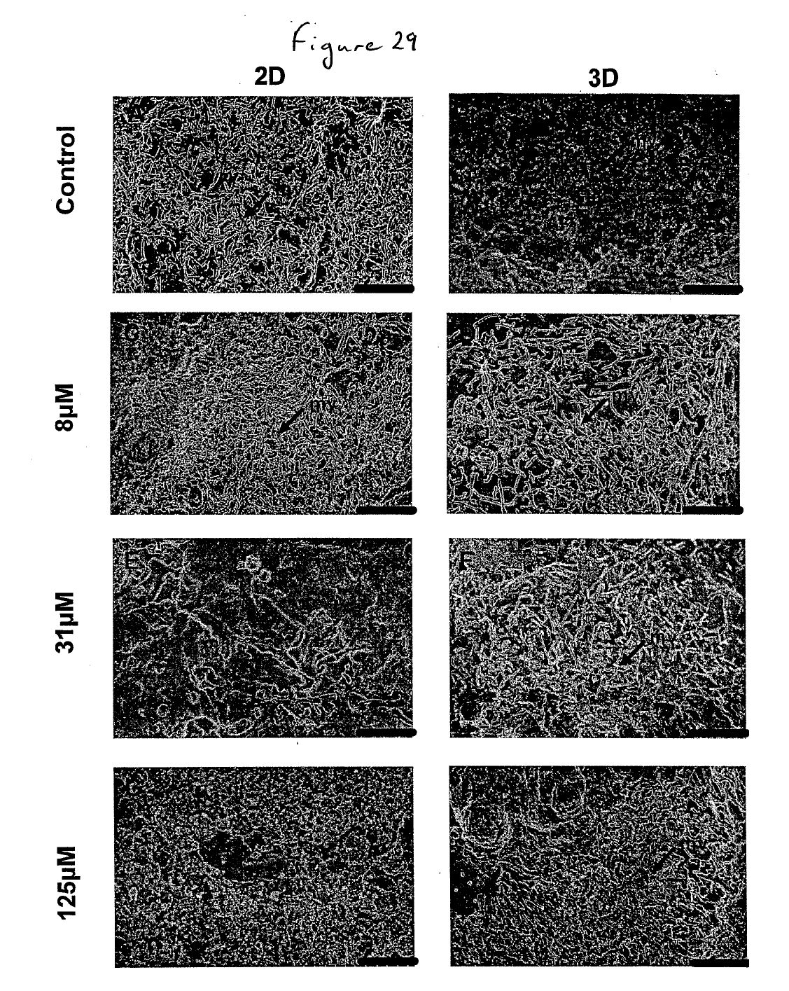

FIG. 29: Scanning electron micrographs showing the effect of methotrexate (MTX) on the surface structure of HepG2 cells. Image panels show HepG2 cells were cultured on 2-D (A,C,E,G) and 3-D (B,D,F,H) substrates, treated with either vehicle (control, no MTX, (A,B)), 8 μM (C,D), 31 μM (E,F), or 125 μM (G,H) MTX. Note that micro-villi (mv) on the cell surface are clearly visible in both control cultures (A,B) and cells exposed to low concentrations of MTX (C,D) when grown on either 2-D (A,C) or 3-D (B,D) substrates. At higher concentrations of the cytotoxin, cells grown on 2-D substrates possessed very few micro-villi (E) and the cell surface showed evidence of breaking up at the maximum levels of MTX tested (F). In contrast, HepG2 cells grown in 3-D and exposed to increasing levels of MTX remained intact and exhibited large numbers of micro-villi (F,H). Scale bars: A-H 2 μm.

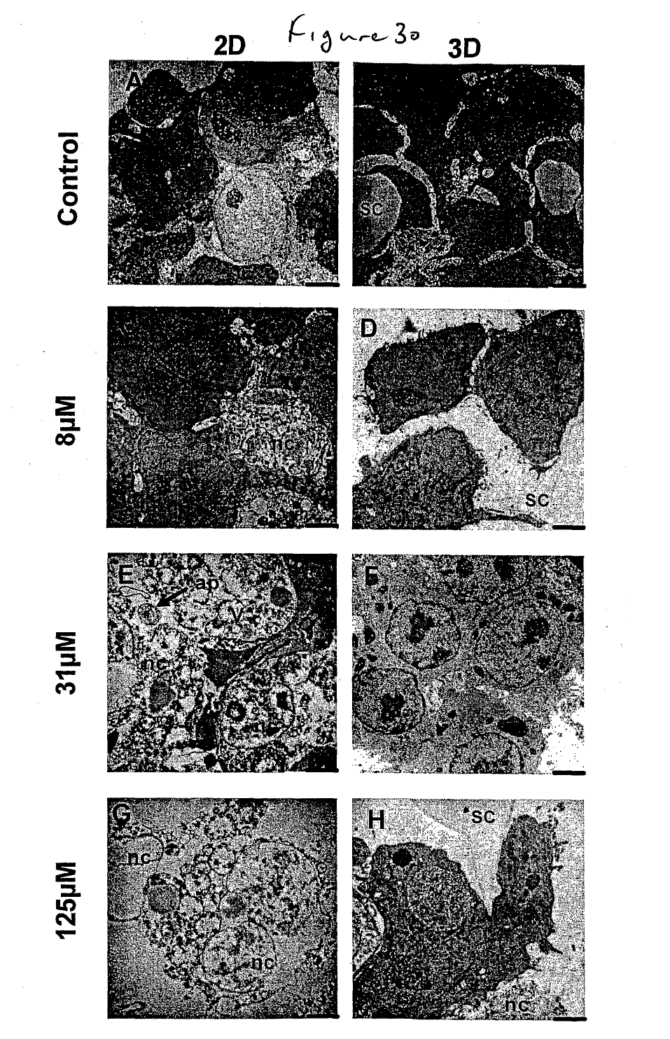

FIG. 30: The effect of methotrexate (MTX) on the ultra-structure of HepG2 cells. Micrographs show cultured cells on 2-D (A,C,E,G) and 3-D (B,D,F,H) substrates, treated with either vehicle (control, no MTX, (A,B)), 8 μM (C,D), 31 μM (E,F), or 125 μM (G,H) MTX. Images of control cultures show the normal structure of cells corresponding to the growth substrate (A,B). The majority of cells grown on flat tissue culture plastic and exposed to 8 μM MTX possessed near normal cellular architecture although a few necrotic cells were identified (C, nc). Increasing concentrations of MTX resulted in the destruction of the vast majority of cells grown on 2-D substrates (E,G). Nuclear membranes had disintegrated and organelles normally found in healthy cells could be identified. There was an increased presence of large vacuolar spaces (v) and membranous bodies known as autophagolysosomes (ap) (E). In contrast, HepG2 cells grown on 3-D scaffolds maintained their structure and only a small number of necrotic cells (nc) were identified in cultures exposed to the 125 μM MTX (H). Scale bars A-H 2 μm.

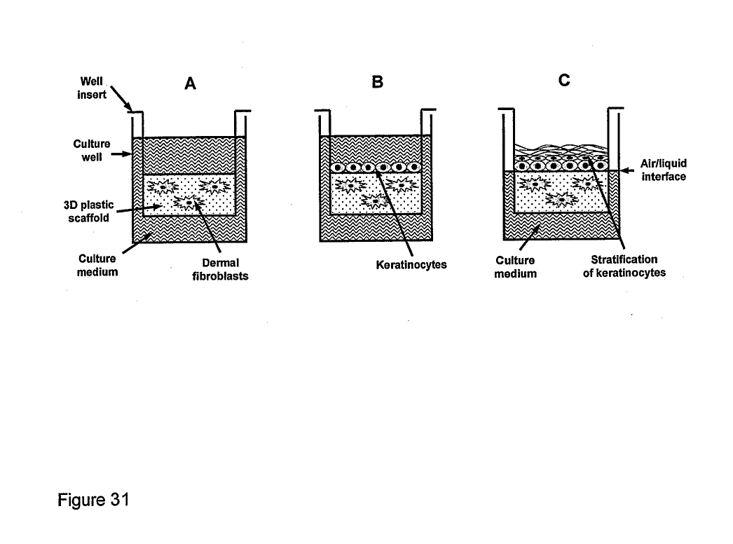

FIG. 31: Example configuration for organotypic coculture of mammalian skin epithelial cells. (A) Well insert with 3D porous polystyrene scaffold attached to base, located in culture well of multi-welled dish (e.g. 6-well plate). Dermal fibroblasts grow within 3D polystyrene scaffold in the presence or absence of collagen gel. (B) Keratinocytes (e.g. HaCaT cells) seeded onto surface of dermal fibroblast culture. (C) Exposure of epidermal keratinocytes to air induces cell stratification achieved in this case by lowering level of culture medium. Cells grown on the 3D scaffolds are readily transferable between different cell culture vessels allowing improved handling by the user.

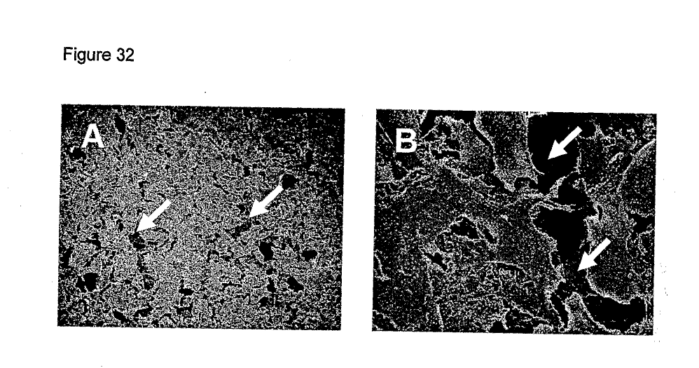

FIG. 32: Scanning electron micrographs of dermal fibroblasts grown on 3D polystyrene scaffolds shown at low (A) and high (B) magnifications. Arrows indicate exposure of the scaffold beneath layer of cells. Structural support of cells improves handling of cultures for routine manipulations by users.

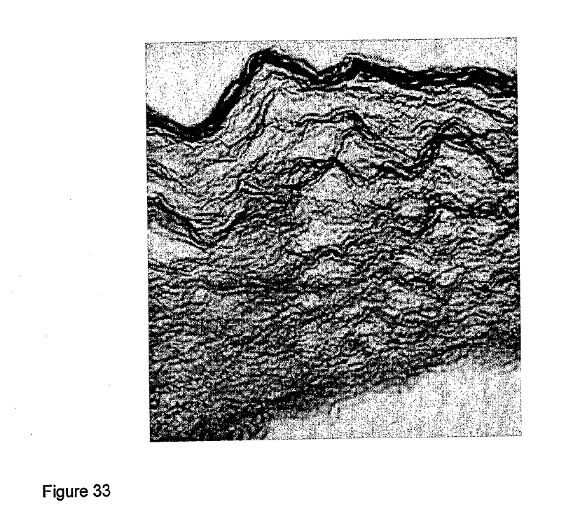

FIG. 33: Stratification of human keratinocytes (HaCaT cells) in organotypic cocultures with fibroblasts grown in 3D. Preparation prepared for histological analysis, sectioned, and epithelial cells stained with Hematoxylin and Eosin.

Table 1 Morphological Parameters of PolyHIPEs Prepared with Different Aqueous Phase Temperatures and Water-miscible Additives;

Table 2 Average Void and Interconnect Diameters of PolyHIPEs Prepared with Aqueous Phase Additives, and Water Self-diffusion Coefficient Values in the Parent HIPEs^{a}; and

Table 3 Influence of Surfactant Concentration on Morphology of PolyHIPEs Prepared with Aqueous Phase Additives.

### Materials and Methods for the Production of Growth Substrate for Routine Use in Cell Culture

Materials Divinylbenzene (Aldrich; 80 vol % divinylbenzene, the remainder being m- and p-ethylstyrene), 2-ethylhexyl acrylate (Aldrich; 99%) and styrene (Aldrich; 99%) were passed through a column of basic activated alumina (Aldrich; Brockmann 1) to remove any inhibitor (4-tert-butylcatechol for styrene and divinylbenzene and hydroquinone or monomethyl ether hydroquinone for 2-ethyhexyl acrylate). Potassium persulfate (Aldrich), sorbitan monooleate (SPAN 80, Aldrich), poly(ethylene glycol) (Aldrich, Mₙ=300) and calcium chloride dihydrate (Aldrich) were used as supplied.

Preparation of PolyHIPE Polymers and Fabrication into Thin Membranes for Cell Culture

PolyHIPE foams were prepared using the polymerisation of a HIPE.
The oil phase contained 60% styrene, 30% 2-ethylhexyl acrylate, 10% divinylbenzene and 25% surfactant (sorbitan monooleate) (all % are w/w).
The aqueous phase contained 1% potassium persulphate in de-ionised H₂O.

### Method

1. In brief, the oil phase was placed in a 3-necked 250 mL round-bottomed flask, fitted with an overhead stirrer (glass rod fitted with a D-shaped PTFE paddle), a 100 mL pressure equalizing dropping funnel (inserted into a side-neck) and a rubber septum. The mixture was purged with nitrogen gas for 15 min.
2. The aqueous phase was heated up to a temperature of 80° C. using a stirrer hotplate and then added to the oil phase over a period of 2 minutes at a constant rate. The emulsion was then mixed for a further minute.
3. The emulsion was then removed and cast in a 50 ml polypropylene tube and left to cure at 60° C. overnight.
4. The polymer was then removed from the tube after 24 hrs and washed extensively in a soxhlet with water and isopropyl alcohol for 24 hrs each.

### Production of Thin Membranes

The polymers were engineered into 120 micron thick membranes. This can be achieved using a microtome or vibrotome should thicker sections (up to 1 mm) be required. Membranes of polymeric material were then sterilized using absolute ethanol, hydrated through a series of graded ethanol solutions and subsequently washed (×3) with sterile phosphate buffered saline (PBS) prior to use. Membranes can be mounted directly into the bottom of existing cell culture plastic-ware (e.g. 6-welled plate) or adhered to a cell culture well insert (see FIGS. 12 and 13).

### Scanning Electron Microscopy

The morphologies of the materials were investigated using a FEI XL30 ESEM operating at between 20-25 kV. Fractured segments were mounted on carbon fibre pads and attached to aluminium stubs and were gold coated using an Edwards Pirani 501 sputter coater. The calculation of average void size was performed using the image analysis software Image J (NIH image). Average diameters measured in this way are underestimates of the real values. Therefore it is necessary to introduce a statistical correction¹. This is achieved by evaluating the average of the ratio R/r, where R is the equatorial value of void diameter and r is the diameter value measured from the micrograph. The statistical factor is calculated from eq. (1).

h²=R²−r² (1)

The probability that the sectioning takes place at any distance (h) from the centre is the same for all values of h, so the average probability value of h is R/2. Replacing this value in eq. (1) gives R/r=2/(3^{1/2}). Multiplication of the observed average value of the void diameter allows a more accurate value to be obtained.

### Mercury Intrusion Porosimetry

Mercury intrusion porosimetry analysis was performed using a Micromeritics AutoPore III 9420. Intrusion and extrusion mercury contact angles of 130° were used. Penetrometers with a stem volume of 1.836 mL and a bulb volume of 5 mL were used. The intrusion volume always comprised between 45 and 80% of the stem volume. Intrusion pressures for the PolyHIPEs never exceeded 200 psi.

### ¹H NMR Diffusion Experiments

The self diffusion coefficient of water (D_{w}) was measured using a 500 MHz Varian Unity Inova 500 narrow bore spectrometer equipped with a Performa II gradient pulse amplifier and an actively shielded 5 mm indirect direction probe. Automated z gradient shimming based on deuterium spin echoes was used. The temperature used for all measurements was 25+/−0.1° C. Water diffusion coefficients were measured using a pulse sequence incorporating pulsed-field gradients such as the bipolar pulse pair stimulated echo (BPPSTE) pulse sequence. Diffusion coefficients are obtained from BPPTSE spectra by monitoring signal attenuation as a function of the applied magnetic field gradient amplitude and fitting eq. (2) to the experimental results.

I=I₀exp[−D(γδG)²(Δ−(δ/2)−(τ/3))] (2)

In eq. (2), I is the resonance intensity measured for a given gradient amplitude, G, I₀ is the intensity in the absence of the gradient pulse, γ is the gyromagnetic ratio, δ is the duration of the bipolar gradient pulse pair, Δ is the diffusion delay time and τ is a short gradient recovery delay time during which relaxation and spin-spin coupling evolution are not significant.

### Hepatocyte Cell Culture

The human hepatic carcinoma cell line, HepG2, was obtained from the American Type Culture Collection (ATCC). HepG2 cells were cultured at 37° C. in 5% CO₂ in growth medium (Dulbecco's modified Eagle medium (D-MEM, Gibco/BRL) supplemented with 10% (v/v) fetal calf serum (FBS, Gibco/BRL), 100 μg.mL⁻¹ penicillin and 10 μg.mL⁻¹ streptomycin (Gibco/BRL)). Cells were passaged every 5-7 days. Confluent cultures of cells were washed with PBS, detached using trypsin/EDTA solution and cell number determined using a hemocytometer. Suspensions of HepG2 cells were then seeded at equal densities either directly into wells of a standard 6-welled plate (Nunc) or into modified well-inserts mounted with the polymer and located in wells of a 6-well plate. Cultures were maintained in growth medium which was changed every 3-4 days or as required.

### Determination of Viable Cell Number

The number of viable cells was determined using a commercially available calorimetric assay (Promega) based on Mosmann's original method for measuring cell activity involving the conversion of a tetrazolium salt into a blue formazan product detectable by a spectrophotometer (570 nm) [32]. The assay was performed according to the manufacturer's instructions on HepG2 cells cultured on 2-D and 3-D substrates for various periods under alternative growth conditions.

### Methotrexate (MTX) Toxicity Studies

Cells were seeded on 2-D and 3-D surfaces in triplicate and left to settle and adhere for 24 hours. The medium was then changed and replaced with medium containing different concentrations of MTX (no MTX (vehicle alone, control), 8 μM, 31 μM, and 125 μM). Cells were subsequently incubated for 1, 3, 7 or 10 days, after which cultures were sampled and assayed for cell number/viability and levels of albumin and transglutaminase were determined.

### Hepatocyte Metabolic Activity

The production of albumin is often used as an indicator of hepatocyte metabolic activity. Levels of albumin were determined using a commercially available kit (Bioassay systems) based on an established method that utilizes bromocresol green which forms a coloured complex specifically with albumin that is detectable at 620 nm. Known quantities of human albumin were used to establish the standard curve. Specific levels of albumin secretion were normalized to total protein levels (as determined by a standard Bradford assay).

### Preparation of Samples for Scanning Electron Microscopy (SEM) and Transmission Electron Microscopy (TEM)

In preparation for SEM, cells grown on 2-D or 3-D substrates were fixed in 2% paraformaldehyde and 2.5% glutaraldehyde in Sorenson's phosphate buffer for 1 hour at room temperature. Samples were then rinsed in 0.1M phosphate buffer and immersed in 1% OsO₄ (aq.) solution for 1 hour, then dehydrated in 50%, 70%, 95% and 100% ethanol for 5 minutes, four times for each respective ethanol change. Samples of fixed 2-D and 3-D cultures were then cut into smaller pieces (approximately 25 mm²), mounted on specimen holders and dried from CO₂ at 38° C. at 1200 psi. The samples were then sputter coated with a 7 nm layer of chromium and examined using a Hitachi S5200 SEM.

For TEM analysis, cells grown on 3-D substrates were fixed and treated as described above for SEM. However, subsequent to dehydration and being cut into small pieces, samples were embedded in resin (Araldite CY212, Agar Scientific) for 1 hour at 37° C. and then placed into pyramidal moulds at 60° C. overnight. For the preparation of cells grown on 2-D surfaces, cultures were fixed in 2% paraformaldehyde and 2.5% glutaraldehyde in Sorenson's phosphate buffer for 1 hour at room temperature. Cells were then scrapped from tissue culture plastic and pelleted at 15,000 rpm for 10 min. Pelleted cells were subsequently rinsed in 0.1M phosphate buffer and immersed in 1% OsO₄ (aq.) solution for 1 hour, then dehydrated in 50%, 70%, 95% and 100% ethanol for 5 min, for times for each respective ethanol change. The dehydrated cell pellets were then soaked in resin (Araldite CY212) for 60 min at 37° C. When set, ultra thin sections of the resin embedded material were produced and subsequently imaged by TEM (Hitachi H7600).

### Enzymatic Assay of Transglutaminase

Tissue transglutaminase is a cross-linking enzyme which has recently been suggested to play a role in the formation of fibrotic lesions in experimental settings. The leakage of this enzyme is often used as a marker for in vitro toxicity testing and its presence indicates damage to cell membranes. Several in vivo and in vitro experimental model systems show a direct relationship between the expression and activity of tissue transglutaminase, suppression of cell growth and programmed cell death [33-35]. The level of transglutaminase was analysed by means of a quantitative enzymatic assay (Sigma, UK) as previously described [36].

### Statistical Analysis

Experiments were performed as at least three independent replicates. Data were analysed for statistical significance using the Mann Whitney U test (at the 5% level of significance or greater).

### EXAMPLE 1

### Effect of Aqueous Phase Temperature

Increasing the temperature of the aqueous phase was found to cause a striking increase in both the average interconnect and void diameter of the PolyHIPE material (FIG. 2). Average void diameters were calculated from a set of 50 voids, the diamaters of which were determined by image analysis of the SEM micrographs. The calculated average void diameter values (<D>) show a steady increase with increasing aqueous phase temperature (Table 1), which is most likely due to the decreasing HIPE stability as the aqueous phase temperature is increased. Increasing the temperature of the aqueous phase, and therefore thermal agitation of the water droplets, will increase the frequency of contact and will result in a higher probability of droplet coalescence². Lissant also reported that, as the emulsion is subjected to heating, the surfactant in the interfacial film separating the droplets becomes more soluble in the bulk liquid phase and therefore migrates from the interface³. This will raise the interfacial tension and thus promote droplet coalescence. It is also noticeable that, as the aqueous phase temperature is increased, the viscosity of the HIPE decreases suggesting that the droplets have a higher mobility. This also helps to promote coalescence.

It has been described in the literature⁴ that the droplet size distribution for an emulsion undergoing coalescence contains the presence of a tail extending towards larger droplet sizes with the maximum staying relatively unchanged. The void size distribution plot (FIG. 3) shows the distribution tailing towards larger void sizes. The tail increases with temperature and an increased broadening of the distribution is also observed. This therefore reinforces the opinion that coalescence is the main mechanism of emulsion instability as the aqueous phase temperature is increased.

The differential plot of intrusion versus interconnect diameter (FIG. 4) shows an increase in interconnect size as the aqueous phase temperature is increased. The plot also shows that, as the aqueous phase temperature is increased, a material with a narrower distribution at higher interconnect diameters and a tail extending in the lower interconnect diameter range exists. This suggests that, for each emulsion, a limiting interconnect diameter exists. This is in contrast to the void size distribution, where a broader distribution is obtained as the temperature is increased. The ratio of the average interconnect (<d>) and void diameters (<D>) provides a measure of the degree of interconnection. The values for the materials prepared in this study are shown in Table 1. As the temperature is increased, the degree of interconnection (<d>/<D>) of the PolyHIPE material decreases. This suggests that, as the aqueous phase temperature is increased, the emulsion stability decreases.

### EXAMPLE 2

### Effect of Additives

Emulsion partial destabilisation can be induced by the presence of organic additives in the aqueous phase. These additives should be partially soluble in both the continuous and the internal phase of the emulsion, which can thus enhance diffusion of water molecules from droplet to droplet and promote Ostwald ripening. Lissant reported³ that addition of co-solvents, such as acetone or methanol, can disrupt the interfacial film due to their solubility in both phases. These additives may dilute the interfacial layer and cause some of the surfactant to migrate into the bulk phase, therefore promoting coalescence of the emulsion droplets.

Poly(ethylene glycol) of Mₙ=300 (PEG₃₀₀), methanol and tetrahydrofuran (THF) were chosen as water-miscible organic species, with a view to selecting species with a range of molar masses and of different polarities. Each component was added to HIPEs in increasing quantities until phase separation occurred. It was found that the emulsion could accept much higher quantities of methanol than either THF or PEG, and this is particularly apparent if one considers the molar quantities of each (0.1 mol methanol, 0.02 mol THF and 0.005 mol PEG). This is due to the greater partitioning of methanol into the aqueous phase, at least in comparison to THF. The octanol/water partition coefficient value (log P_{ow}) for THF is 0.45, whereas that of methanol is −0.77. No log P_{ow} value could be found for PEG. The SEM images (FIG. 5) suggest that each additive produces an increase in the average void and interconnect size. Image analysis of the SEM micrographs produces the void size distributions, which are shown in FIG. 6. FIG. 6a shows that the addition of PEG produces a material with a wider distribution of void sizes than the PolyHIPE material with no additive present, with a tail extending towards larger void sizes. This pattern is similar to that obtained for methanol (FIG. 6b) and THF (FIG. 6c), however with methanol the effect on the distribution is less than that for THF or PEG. The materials prepared with THF or PEG contain a wider range of void sizes than the materials prepared with methanol. PEG and THF also produced PolyHIPE materials with higher average void size values (see Table 1).

The interconnect distribution curves are similar in nature for all additives used (FIG. 7). As the concentration of additive is increased, there is a tendency towards materials with a higher average interconnect diameter and a narrower size distribution. The exception to this trend is when THF is used as the additive. In this case a broad distribution is still obtained at high THF concentration. When PEG was used as the additive, the average interconnect diameter values increased steadily with PEG concentration in the aqueous phase (Table 1). This effect was not as pronounced for THF or methanol; for these additives, much higher concentrations were needed to produce a significant change in the interconnect diameter. In the case of THF, this was unexpected as it had the most significant effect on the average void diameter. The degree of interconnection (<d>/<D>) decreases following initial addition of the organic component, which is brought about by the large increase in void diameter compared to interconnect diameter. After the initial addition of PEG or methanol, the degree of interconnection increases steadily with the concentration in the aqueous phase. However, in the case of THF, the degree of interconnection continued to decrease with increasing concentration. This is because THF has no significant effect on the interconnect size until the concentration reaches 1.5%.

In previous work⁵ the increase in void size of PolyHIPE materials on addition of a co-solvent or additive has been ascribed solely to Ostwald ripening. However, it is possible that organic additives influence other processes that lead to emulsion destabilisation. As discussed previously, the addition of a co-solvent can disrupt the interfacial layer by causing surfactant to migrate into the bulk phase, causing the emulsion droplets to become more prone to coalescence. It is possible that the rate of both coalescence and Ostwald ripening are enhanced by the addition of co-solvents to the system, and that one process may dominate depending on the exact system (emulsion type, surfactant type, etc.).

In order to probe the influence of Ostwald ripening, the self diffusion coefficient of water, D_{w}, was monitored in the presence of each additive over a period of 8 hours (Table 2). When methanol is present there is no significant difference in the diffusion coefficient compared to the emulsion with no additive present. When PEG and THF are used, there is a significant effect on the diffusion coefficient with a greater effect observed for THF. These values can be correlated with the average void and interconnect diameters obtained.

The change in the diffusion coefficient of water over time (ΔD_{w}) can give a possible insight into the effect of each co-solvent on the emulsion (FIG. 8). In the presence of THF and PEG, there is greater increase in D_{w} compared to the emulsion with no co-solvent present. This suggests that both THF and PEG enhance the rate of water diffusion in the emulsion, which would increase Ostwald ripening and is a possible explanation for the increase in void size. The octanol/water partition coefficient value (log P_{ow}) for the additives (THF=0.45; methanol=−0.77) indicates that THF partitions more into the oil phase than methanol, resulting in a less stable emulsion in the presence of THF.

Since there was no significant increase in D_{w} in the presence of methanol, other effects such as coalescence must be taken into account to explain the observed increase in void diameter. Coalescence can be promoted by dilution of the interfacial layer and subsequent migration of surfactant into the bulk phase due to its increased solubility in the presence of the co-solvent. If this is the case, the surfactant concentration (Cₛ) should have an effect on the final morphology of the material.

To investigate the influence of Cₛ on morphology, PolyHIPE materials with different Cₛ values were prepared using THF and methanol as additives. Since THF had been shown to enhance water diffusion, this would allow us to investigate whether the morphology obtained with THF was solely due to Ostwald ripening or whether surfactant depletion from the interface was also involved. Methanol, on the other hand, was shown to have no influence on the rate of water diffusion. Therefore, it was expected that an increase in Cₛ would have a profound effect on morphology if methanol was influencing the surfactant concentration at the interface. It can be observed from the SEM images (FIG. 9a, b) that, as the surfactant concentration in THF containing HIPEs is increased, there is an increase in the open nature of the material but no real discernible effect on void diameter. From the void distribution chart, however, there is a small shift to lower void diameter with increasing surfactant concentration (FIG. 10a). However, no flattening or broadening of the distribution is observed with decreasing surfactant concentration.

With THF as additive, the <d>/<D> value (Table 3) increases as the surfactant concentration is increased. This is caused by a slight reduction in <D> with little effect on <d> and provides further evidence that, as the surfactant concentration is increased in the presence of the THF, the open nature of the material increases. However, although there is a slight decrease in the average void size with increasing surfactant concentration, there is a still a significant increase in <D> relative to the material prepared with no additive present (compare entry 3 in Table 3 with entry 1 in Table 1). This suggests that Ostwald ripening is the dominant effect in determining the morphology of polystyrene-based PolyHIPE materials prepared in the presence of THF.

The surfactant concentration was also increased in the presence of methanol in the aqueous phase. This had little effect on the morphology of the resulting materials (FIG. 9c, d). From the void size distribution plots (FIG. 10b) there is little difference in the distribution when the surfactant concentration is increased from 20 to 30% w/w. The only discernible effect occurs when the surfactant concentration was increased to 30% w/w, at which there is a small increase in the percentage of voids present with a diameter of between 30 and 40 μm and a decrease in the percentage of voids present at higher diameters. However, Table 3 indicates that a maximum value of <D> is obtained when C_{S}=25%.

The surfactant concentration has little effect on the interconnect diameter when THF is used as the additive (FIG. 11a). In contrast, increasing the surfactant concentration from 20 to 25% w/w results in a peak shift towards larger interconnect diameters (FIG. 11b) in the presence of methanol. However, when the surfactant concentration is increased from 25% to 30% w/w smaller interconnect diameters are obtained, although the <d>/<D> ratios (Table 3) for both materials are similar since this is also accompanied by a decrease in <D>.

From these results, we conclude that Ostwald ripening is not the cause of the increased void diameter in the presence of methanol, since this additive has no influence on the rate of water self-diffusion. Another process by which water can be transported from droplet to droplet is in the interior of w/o micelles⁶, which are known to be present in the continuous phase of HIPEs⁷. An increase in surfactant concentration would increase the number of micelles in the continuous phase, which could enhance water transport between droplets. To explain the results obtained with methanol when the surfactant concentration is increased, we conclude that the added surfactant influences two opposing processes: it replaces the surfactant depleted by methanol, which stabilises the emulsion; and it also increases the number of w/o micelles, enhancing water transport and destabilizing the emulsion. Support for this comes from the observed maximum values of <D> and <d> at Cₛ=25%, suggesting that two independent processes are operating. The net effect is that there is little observed change in the morphology of PolyHIPEs prepared with methanol when the surfactant content is increased from 20 to 30% (w/w).

In conclusion, it has been shown that different methods can be used to control emulsion stability to produce PolyHIPE materials with a wide range of void and interconnect sizes. Controlling these parameters will allow the production of different scaffold structures, each tailored and customised toward use in the 3D culture of different cell types.

### EXAMPLE 3

The ability to control the structure of polyHIPE materials is critical to ensure the optimal growth of cultured cells in a 3-dimensional fashion. We have developed a novel application of these materials for this purpose by engineering styrene-based polymeric scaffolds into thin membranes suitable for routine cell growth in vitro by adapting their use to existing tissue culture plastic-ware (see examples: FIGS. 12 and 13). The approach to using thin layers with large surface areas allows: (1) Good access of the cells into the structure of the material by either static or dynamic seeding; allows good access of oxygen and nutrients (in some cases from both sides of the membrane—see FIG. 12, example 1) and removal of waste materials and carbon dioxide therefore minimising the chance of necrosis occurring as found in polymer scaffolds of larger dimensions. These attributes promote the viability of the cultured cells in 3-dimensions; (2) Good access by exogenous reagents (for example, test compounds) to cells growing in 3-dimensions; (3) Cells to be removed from the scaffold after 3-dimensional cell growth for further analysis using enzymatic treatments such as incubation with trypsin (data not shown); (4) Cells and tissues growing within the scaffold may be visualised using electron or optical microscopy (FIGS. 14 and 20. respectively).

The ability to control the dimensions that constitute the structure of the polymeric material is essential for optimisation of cell growth and behaviour. Subtle differences in the porosity of these materials have significant implications on the ability of cells to adhere to the scaffold, proliferate, differentiate and function (FIGS. 15-17). Styrene-based polymeric materials produced with 90% pore volume and optimised for in vitro cell growth also enhance cell proliferation, differentiation and function compared to conventional 2-dimensional cell culture plastic-ware (FIGS. 18-24).

### EXAMPLE 4

### Morphological Characteristics of HepG2 Cells Grown on Alternative Substrates

Scanning electron microscopy revealed significant differences in the appearance of HepG2 cells cultured either on 2-D or 3-D substrates (FIG. 25). Cells grown on 2-D planar surfaces formed flat extended structures after 7 days. In general, 2-D cultures appeared heterogeneous and disorganised. After 14 days, cells cultured on tissue culture plastic started to cluster and form aggregates. In some areas of 2-D cultures grown for 14-21 days, HepG2 cells appeared unhealthy, some were rounding up and others were disintegrating (data not shown). Cells cultured on 3-D polystyrene, spread across and into the structure of the scaffold. Cells initially clustered into colonies of closely packed cells within the substance of the polymer, resembling small multi-cellular aggregates. This was indicative of the greater attachment and interaction between adjacent cells growing on the scaffold. After 7 days, cultures of HepG2 cells grown in 3-D appeared more homogeneous than their 2-D counterparts. Growing cultures at lower seeding densities showed that cells attached to the scaffold and extended across voids. This demonstrated how cells grown in 3-D can maximise their surface area by interacting with adjacent cells and the incubation medium. In addition, higher magnification imaging of individual cells grown in 3-D revealed a significantly greater number of micro-villi compared to cells grown on 2-D surfaces.

Transmission electron microscopy was used to examine the ultra-structural features of HepG2 cells cultured on different materials (FIG. 26). In general, analysis of intact whole cells grown on either 2-D or 3-D substrates contained a range of organelles typical of most mammalian cells, including mitochondria, nuclei, endoplasmic reticulum and lipid droplets. Ultra-thin sections of cell preparations grown in 3-D showed clearly how cells grow around and in close association with the polystyrene scaffold. Cells cultured on the 3-D surfaces displayed numerous morphological features typical of the liver tissue. Nuclear membranes appeared to be normal. Numerous mitochondria visualised displayed structural variances within the normal range. No specific pathological alterations were detectable in either the smooth or the rough endoplasmic reticulum, in the Golgi complexes, or in the glycogen content. The presence of these ultra-structural features indicated that HepG2 cells grown on 3-D substrates were metabolically active. The presence of peroxisomal clusters (FIG. 26B), which are ubiquitous cell organelles abundant in mammalian liver and kidney, was particularly encouraging. Liver peroxisomes are known to be responsible for the β-oxidation of the side chain of cholesterol in the course of bile acid synthesis, a pathway associated with differentiated hepatocytes [8].

In native liver tissue, hepatocytes possess polarity with two or three basal surfaces facing the sinusoid while adjacent cells form the bile canaliculi. Micrographs of cells grown in 3-D revealed adjacent hepatocytes often shared microvilli-lined channels lined with tight junctions. This observation suggests that cultured HepG2 cells may be polarized and capable of forming channels that resemble bile canaliculi [9]. These structures are known to be rich in microvilli and components of bile metabolised in the cells are normally secreted into the canaliculi.

### EXAMPLE 5

### Enhanced Cell Viability During 3-D Cell Growth

We examined whether the surfaces used in this study were biocompatible to support the growth of viable HepG2 cells. FIG. 27A illustrates the MTT absorbance values for hepatocytes grown on 2-D control surfaces and our 3-D scaffolds. Viable cells were successfully cultured on both substrates for up to 21 days. The assay revealed that cell viability was significantly enhanced when grown in 3-D. It should be noted that cells grown on scaffolds have a greater surface area on which to attach and grow, compared to planar surfaces, where space per cell is restricted. Where possible, this difference has been taken into account and values were normalized for in vitro assays.

### EXAMPLE 6

### Enhanced Cell Metabolism During 3-D Cell Growth

The metabolic activity of HepG2 cells was assessed by determination of the level of albumin secretion. FIG. 27B shows the time courses of albumin secretion on 2-D tissue culture plastic and the 3-D scaffolds. Values have been normalized to account for any differences in cell number. It can be seen clearly that there is a significantly higher albumin concentration in cultures grown on 3-D surfaces compared to cells grown in 2-D for all the time points that were tested. Albumin levels in cultures grown on flat tissue culture plastic peaked at day 14 and then decreased rapidly at 21 days. This did not occur in cultures grown on 3-D scaffolds indicating that the 3-D environment is more conducive to cell function.

### EXAMPLE 7

### Effects of Methotrexate on Cells Grown in 2-D and 3-D

HepG2 cells grown in 2-D and 3-D formats were treated with various concentrations of MTX to evaluate their tolerance to a well known cytotoxin. Following each treatment period, cultures were then studied for biochemical (FIG. 28) and morphological changes (FIGS. 29 & 30).

FIG. 28A illustrates cell viability after 1 and 7 days treated with varying concentrations of MTX. Treatment of HepG2 monolayers with MTX resulted in a gradual increase of absorbance at 15 μM MTX after 24 hours but absorbance levels started to drop at 7 days (data not shown). With increasing MTX concentrations, the viability of cells grown on 2-D surfaces was visibly reduced especially at the higher levels of the cytotoxin. In 3-D cultures, sensitivity to MTX was not evident in the lesser concentrations of MTX; only at 62 μM MTX was there a significant decrease in absorbance levels compared to control values. This pattern was also seen in 3-D cultures grown for 7 days, whereas cells grown in monolayers for 7 days showed a sharp decrease in absorbance levels at 125 μM MTX concentrations. These results imply that cells cultured on 2-D surfaces under these conditions remain viable for a shorter period compared to cells cultured on 3-D scaffolds.

The metabolic activity of HepG2 cells was significantly reduced by increasing concentrations of MTX (FIG. 28B). Cells grown in 2-D were sensitive to the lowest concentrations of MTX tested (8 μM), whereas this level did not significantly influence albumin secretion by cells grown in 3-D (data not shown). However, increased levels of MTX (15.6 μM) did begin to reduce albumin secretion by cells cultured on scaffolds. Throughout cytotoxic challenge, higher levels of albumin secretion were noted in cells grown on 3-D plastic compared to 2-D materials. These data illustrate that hepatocyte cell function was impaired in the presence of MTX in a dose-dependent manner and cells grown in 3-D appeared more tolerant to the cytotoxin.

Measurement of transglutaminase was performed as a test for HepG2 cell toxicity in response to increasing concentrations of MTX. We examined the effects of MTX on transglutaminase levels in 2-D and 3-D cultures after 1, 7 and 10 days exposure to the cytotoxin. (FIG. 28C). In control cultures, where there was no addition of MTX, levels of transglutaminase were found to be minimal and at similar levels. With increasing concentrations of the drug, such as 31 μM MTX, 2-D cultures secreted significantly higher levels of transglutaminase which increased in a dose dependent manner unlike cells grown in 3-D culture. These differences were statistically significant in all 2-D cultures at 7 and 10 days compared to their 3-D counterparts. In the 3-D cultures, increasing the concentration of MTX did not cause a significant increase in transglutaminase levels although there was a gradual rise in enzyme levels secreted into the culture medium at higher concentrations of the drug. These data further suggest that cells on 3-D porous materials are more tolerant to increasing levels of cytotoxin challenge.

Scanning (SEM) and transmission electron micrographs (TEM) demonstrated concentration dependent changes in cell structure subsequent to treatment with MTX. Representative examples of such morphological changes are illustrated in FIGS. 29 and 30. Normal, healthy HepG2 cells express numerous micro-villi on their cell surface. When challenged with MTX, cells grown on 2-D surfaces gradually lost their micro-villi in a dose dependent manner, whilst cells grown on scaffolds continued to express this structural feature (FIG. 29). In response to increasing levels of MTX, the surface of HepG2 cells grown as monolayers, first decreased the numbers of micro-villi, then became flattened, and then started to disintegrate. No such changes were observed to the structure of HepG2 cells grown on 3-D scaffolds, although the micro-villi of cells grown in the highest concentration of MTX tested (125 μM), did begin to show signs of flattening.

Further examination by TEM revealed ultra-structural changes to cells challenged by the cytotoxin (FIG. 30). Cells grown on 2-D surfaces possessed healthy morphology with prominent nuclei with visible nucleoli in control cultures. Following treatment with 8 μM MTX, cells with good nuclear architecture remained visible in 2-D cultures although areas of cellular necrosis were also evident. Hepatocyte 2-D cultures treated with increasing levels of MTX showed marked cytotoxicity and most cells became necrotic at high levels of MTX. Features such as endoplasmic reticulum de-granulation and the presence of ribosomal ghosts were observed. Furthermore, the granular cytoplasm generally lacked an organised structure and clumped chromatic granules were dispersed throughout the nucleus. Sub-cellular evidence of apoptosis was also observed; the plasma membrane was seen to rupture and a marked amount of vacuolation, possibly reflecting presence of lipid droplets and cellular degeneration. Cell shrinkage was also obvious, as well as a loss of cell-to-cell contact followed by formation of apoptotic bodies (autophagolysosomes) and cell death. At the highest concentration, ‘ghosts’ of cellular remains were observed, indicating that cells grown in 2-D exposed to higher levels of MTX had undergone an advanced stage of cell death.

In contrast, HepG2 cells grown in 3-D culture and exposed to MTX were significantly more resistant to the effects of the cytotoxin. The ultra-structure of cells treated with lower concentrations of MTX possessed normal organelles in their cytoplasm (RER, ribosomes, mitochondria and lipid droplets). The nuclei displayed normal heterochromatin and nucleoli. These features were well preserved throughout most of the concentrations of MTX tested. However, in some cells in the presence of 125 μM of MTX, the nuclear membrane had an irregular morphology and other sub-cellular features, such as mitochondria, which appeared to be slightly abnormal. It is likely therefore at higher concentrations of MTX, cells in 3-D cultures are starting to undergo changes similar to those experienced by cells cultured in 2-D as seen significantly lower concentrations of the cytotoxin.

In conclusion, the growth of cells on styrene-based polymeric scaffolds adapted for use in existing cell culture plastic-ware provides the opportunity for 3-dimensional cell growth in vitro. Cell behaviour is influenced by the environment in which cells grow and cell growth in 3-dimensions is more realistic and more closely resembles the growth conditions cells normally experience in the body. The apparatus described herein provides an opportunity for researchers to routinely grow cells in 3-dimensions which will be invaluable for more accurate read-outs from cell models and assays. The apparatus is also inert, easy to use, can be sterilised, is cheap to manufacture and produce, it is robust and reproducible, has an indefinite shelf-life and is adaptable to many applications.

### EXAMPLE 8

In a further application for the use of the polystyrene scaffold, we have developed an organotypic model of mammalian skin consisting of a stratified sheet of epidermal keratinocytes grown at the media/air interface on a layer of dermal fibroblasts in the presence or absence of a collagen gel or solution-coating within the scaffold. This system enables long term growth and maintenance of polarised epithelia that closely resemble native skin. The technology can be used to investigate the function of skin epithelial cells in a broad range of applications, including basic science, development of pharmaceuticals and assessment of compound toxicity.

Organotypic models for the growth of mammalian skin are well established and a number of procedures have been developed to achieve this in vitro (for example: Bohnert et al. 1986; Ikuta et al. 2006; Prunieras et al. 1983; Schoop et al. 1999). The existing procedures requires the growth of dermal fibroblasts within a collagen gel mixture, upon which keratinocytes are seeded in a two layered sandwich. The gel shrinks over time; it is then raised to the air/media interface to enable changes in cell growth and activity. Handling the gel is tricky and requires time, skill and concentration. As a consequence this model is not readily adaptable for high throughput screening strategies or in circumstances where a reduction in variability is required and ease of handling is needed.

Here we demonstrate the application of our 3D polystyrene scaffolds to more readily enable the routine use and handling of dermal fibroblasts and collagen gels for organotypic skin cocultures. In brief, cultures of dermal fibroblasts are seeded onto the surface of our 3D polystyrene scaffolds in appropriate growth media (FIG. 31a). The cells grow over the surface and into the structure of the 3D membrane (FIG. 32). This can be achieved in the presence or absence of a collagen solution/gel or pre-coating of the scaffold with collagen solution (e.g. Type I collagen). The inert 3D plastic scaffold provides support for the cultured fibroblasts. The scaffolds laden with fibroblasts are readily handled and can be transferred into fresh cell culture plastic ware (e.g. 6-welled plate) if required. In addition, shrinkage of a cast collagen gel is minimised which reduces variability between experiments. Subsequent to the establishing the fibroblast culture, an organotypic coculture is set up by seeding epidermal keratinocytes (e.g. HaCaT cells) onto the surface of the fibroblasts (FIG. 31b). When the keratinocyte culture is established (˜2 days), the surface of the polystyrene scaffold is raised to the air-liquid interface. Air exposure induces stratification of the keratinocytes (FIGS. 31c and 33).

The advantages for using the 3D porous polystyrene scaffolds for organotypic coculture of mammalian skin are:
To provide support for the cells (and gel if appropriate) and 3D environment for the dermal fibroblast culture
To enable ease of handling of the fibroblast culture/collagen gel mix and avoid breakage or damage to the gels/culture
To minimise shrinkage of the collagen gel
To enable freedom to readily transfer organotypic cultures to other vessels
To raise the culture to the air/liquid interface either by reducing the media level or by raising the scaffold itself (for example, using the well insert configuration together with adaptors to increase the height of the insert)

### REFERENCES

1. A. Barbetta and N. R. Cameron, Macromolecules, 2004, 37, 3188.
2. A. S. Kabalnov and E. G. Shchukin, Adv. Colloid Interface. Sci., 1992, 38, 69.
3. K. J. Lissant (ed.), ‘Emulsions and Emulsion Technology Part 1’, Marcel Dekker Inc., New York, 1974.
4. M. P. Aronson and M. F. Petko, J. Colloid Interface Sci., 1993, 159, 134.
5. M. W. Hayman, K. H. Smith, N. R. Cameron, and S. A. Przyborski, Biochem. Biophys. Res. Commun., 2004, 314, 483; M. W. Hayman, K. H. Smith, N. R. Cameron, and S. A. Przyborski, J. Biochem. Biophys. Methods, 2005, 62, 231.
6. J. G. Weers, in ‘Modern Aspects of Emulsion Science’, ed. B. P. Binks, RSC, Cambridge, 1998.
7. J. C. Ravey and M. J. Stebe, Prog. Colloid Polym. Sci., 1990, 82, 218; R. Pons, P. Erra, C. Solans, J. C. Ravey, and M. J. Stebe, J. Phys. Chem., 1993, 97, 12320; R. Pons, J. C. Ravey, S. Sauvage, M. J. Stebe, P. Erra, and C. Solans, Colloids Surf., A: Physicochem. Eng. Aspects, 1993, 76, 171; J. C. Ravey, M. J. Stebe, and S. Sauvage, Colloids Surf., A: Physicochem. Eng. Aspects, 1994, 91, 237.
8. Beier K, Fahimi H D. Application of automatic image analysis for quantitative morphological studies of peroxisomes in rat liver in conjunction with cytochemical staining with 3-3′-diaminobenzidine and immunocytochemistry. Microsc Res Tech 1992 Jun. 1; 21(4):271-282.
9. Abu-Absi S F, Friend J R, Hansen L K, Hu W S. Structural polarity and functional bile canaliculi in rat hepatocyte spheroids. Exp Cell Res 2002 Mar. 10; 274(1):56-67.
10. Bohnert A, Hornung J, Mackenzie I C, Fusenig N E (1986). Epithelial-mesenchymal interactions control basement membrane production and differentiation in cultured and transplanted mouse keratinocytes. Cell Tissue Res 244:413-429.
11. Ikuta S, Sekino N, Hara T, Saito Y, Chida K (2006). Mouse epidermal keratinocytes in three-dimensional organotypic coculture with termal fibroblasts form a stratified sheet resembling skin. Biosci Biotechnol Biochem 70:2669-2675.
12. Prunieras M, Regnier M, Woodley D (1983). Methods for cultivation of keratinocytes with an air-liquid interface. J Invest Dermatol 81:28s-33s.
13. Schoop V M, Mirancea N, Fusenig N E (1999). Epidermal organisation and differentiation of HaCaT keratinocytes in organotypic coculture with human dermal fibroblasts. J Invest Dermatol 112:343-353.

TABLE 1

| Temp_{aq}^{a}/° C. | Additive (%)^{b} | <D>/μm^{c} | <d>/μm^{d} | <d>/<D> |
|---|---|---|---|---|
| 23 | — | 35 | 11 | 0.29 |
| 50 | — | 74 | 16 | 0.27 |
| 60 | — | 94 | 19 | 0.22 |
| 80 | — | 104 | 26 | 0.25 |
| 23 | PEG (0.2) | 82 | 12 | 0.15 |
| 23 | PEG (0.4) | 73 | 14 | 0.19 |
| 23 | PEG (0.8) | 84 | 15 | 0.17 |
| 23 | PEG (1.5) | 74 | 16 | 0.22 |
| 23 | MeOH (1.0) | 59 | 12 | 0.20 |
| 23 | MeOH (2.0) | 57 | 12 | 0.21 |
| 23 | MeOH (3.0) | 68 | 16 | 0.24 |
| 23 | MeOH (4.0) | 65 | 14 | 0.22 |
| 23 | THF (0.4) | 60 | 12 | 0.20 |
| 23 | THF (0.8) | 90 | 12 | 0.13 |
| 23 | THF (1.0) | 72 | 12 | 0.17 |
| 23 | THF (1.5) | 99 | 15 | 0.15 |

^{a}aqueous phase temperature
^{b}aqueous phase additive expressed as vol. % (wt./vol. % for PEG)
^{c}average void diameter determined by SEM
^{d}average interconnect diameter determined by Hg porosimetry

TABLE 2

| | | | (D_{wi}/m²s⁻¹) × | (D_{wf}/m²s⁻¹) × | (ΔD_{w}/m²s⁻¹) × |
|---|---|---|---|---|---|
| Additive (%)^{b} | <D>/μm^{c} | <d>/μm^{d} | 10^{-10e} | 10^{-10f} | 10^{-10g} |
| — | 35 | 11 | 7.1 | 8.2 | 1.1 |
| THF (1.5) | 99 | 15 | 10.1 | 12.1 | 2 |
| MeOH (2) | 57 | 12 | 7.3 | 8.1 | 0.8 |
| PEG (1.5) | 74 | 16 | 7.8 | 9.4 | 1.6 |

^{a}In each case the aqueous phase was kept at room temperature during emulsion preparation.
^{b}aqueous phase additive expressed as % (v/v) (% (w/v) for PEG)
^{c}average void diameter determined by SEM
^{d}average void diameter determined by Hg porosimetry
^{e}D_{wi} = initial value of water self-diffusion coefficient
^{f}D_{wf} = final value of water self-diffusion coefficient
^{g}□D_{w} = change in water self-diffusion coefficient

TABLE 3

| Additive^{a} | Cₛ (% w/w)^{b} | <D>/μm^{c} | <d>/μm^{d} | <d>/<D> |
|---|---|---|---|---|
| THF | 20 | 74 | 12 | 0.16 |
| THF | 25 | 72 | 12 | 0.17 |
| THF | 30 | 66 | 14 | 0.22 |
| MeOH | 20 | 58 | 10 | 0.17 |
| MeOH | 25 | 65 | 15 | 0.23 |
| MeOH | 30 | 53 | 12 | 0.23 |

^{a}1.5% (v/v) THF, 4% (v/v) methanol
^{b}Concentration of surfactant (Span 80) expressed as percentage of total monomer phase
^{c}average void diameter determined by SEM
^{d}average interconnect diameter determined by Hg porosimetry

1. A cell culture substrate comprising a plurality of microcellular polymeric material wherein the pore volume of the microcellular polymeric material is between 88% and 92%.

2. A substrate according to claim 1 wherein the pore volume is about 90%.

3. A substrate according to claim 1 wherein said substrate comprises a hydrophobic elastomer at a concentration of between 20% (w/w) and 40% (w/w).

4. A substrate according to claim 3 wherein said hydrophobic elastomer at a concentration of between 25% (w/w) and 35% (w/w).

5. A substrate according to claim 3 wherein said hydrophobic elastomer is provided at a concentration of 30% (w/w).

6. A substrate according to claim 3 wherein said elastomer is selected from the group consisting of: 2-ethylhexyl acrylate; n-butyl acrylate and n-hexyl acrylate.

7. A substrate according to claim 6 wherein said elastomer is 2-ethylhexyl acrylate.

8. A substrate according to claim 6 wherein 2-ethylhexyl acrylate is provided at between 28% (w/w) and 32% (w/w).

9. A substrate according to claim 8 wherein 2-ethylhexyl acrylate is provided at about 30% (w/w).

10. A substrate according to claim 1 wherein said cell culture substrate comprises polyvinyl.

11. A substrate according to claim 10 wherein said polyvinyl is polystyrene.

12. A substrate according to claim 11 wherein said polystyrene comprises a styrene monomer and divinyl benzene.

13. A substrate according to claim 1 wherein said cell culture substrate comprises a surfactant.

14. A substrate according to claim 13 wherein said surfactant is provided at a concentration of 20-30% (w/w).

15. A substrate according to claim 14 wherein said surfactant is provided at a concentration of between 24-26% (w/w).

16. A substrate according to claim 15 wherein said surfactant is provided at a concentration of around 25% (w/w).

17. A substrate according to claim 1 wherein said cell culture substrate comprises a plurality of membrane or thin layers of microcellular polymeric material wherein said membrane/layer is 50-1000 microns thick.

18. A substrate according to claim 17 wherein said membrane/layer is approximately 120-150 microns thick.

19. A substrate according to claim 1 wherein said microcellular polymeric material comprises a further organic monomer.

20. A substrate according to claim 19 wherein said organic monomer is selected from the group consisting of: n-butyl methacrylate, n-hexyl methacrylate, cyclohexyl acrylate, cyclohexyl methacrylate, phenyl acrylate, phenyl methacrylate, 3-vinylbenzyl chloride, 4-vinylbenzyl chloride, para-acetoxystyrene.

21. A substrate according to claim 1 wherein said microcellular polymeric material comprises a further organic polymer.

22. A substrate according to claim 21 wherein said organic polymer is selected from the group consisting of: poly(n-butyl methacrylate), poly(n-hexyl methacrylate), poly(cyclohexyl acrylate), poly(cyclohexyl methacrylate), poly(phenyl acrylate), poly(phenyl methacrylate), poly(3-vinylbenzyl chloride), poly(4-vinylbenzyl chloride), poly(para-acetoxystyrene).

23. A substrate according to claim 1 wherein said cell culture substrate comprises a surface that has been modified by the provision of a coating that facilitates the attachment, proliferation and/or differentiation of cells attached thereto.

24. A substrate according to claim 23 wherein said modification is the provision of a proteinaceous coating.

25. A substrate according to claim 24 wherein said proteinaceous coating comprises at least one molecule selected from the group consisting of: laminin, collagen, fibronectin, non-collagen based peptide matrices.

26. A substrate according to claim 24 wherein said proteinaceous coating comprises a poly-amino acid coating.

27. A substrate according to claim 26 wherein said polyamino acid coating comprises poly L ornithine or poly L lysine.

28. A substrate according to claim 23 wherein the surface of said cell culture substrate is physically modified.

29. A substrate according to claim 28 wherein said substrate comprises a surface that is modified by gas plasma treatment.

30. A substrate according to claim 29 wherein said surface is modified by a plasma gas treatment comprising ammonia.

31. A substrate according to claim 29 wherein said surface is modified by a plasma gas treatment comprising oxygen.

32. A cell culture vessel comprising a cell culture substrate according to claim 1.

33. A vessel according to claim 32 wherein said cell culture substrate further comprises a cell and cell culture media.

34-45. (canceled)

46. A method for the culture of cells comprising the steps of:
i) providing a cell culture vessel comprising:
a) cells;
b) a cell culture substrate according to claim 1;
c) cell culture medium sufficient to support the growth of said cells; and
ii) providing cell culture conditions which promote the proliferation and/or differentiation of said cells.

47-51. (canceled)

52. A method to screen for an agent wherein said agent affects the proliferation, differentiation or function of a cell comprising the steps of:
i) providing cell culture comprising at least one cell and a cell culture substrate according claim 1;
ii) adding at least one agent to be tested; and
iii) monitoring the activity of the agent with respect to the proliferation, differentiation or function of said cells.

53. (canceled)

54. A method for the identification of genes associated with cell differentiation comprising the steps of:
i) providing cell culture comprising at least one cell and a cell culture substrate according to claim 1;
ii) extracting nucleic acid from cells contained in said cell culture;
iii) contacting said extracted nucleic acid with a nucleic acid array; and
iv) detecting a signal which indicates the binding of said nucleic acid to a binding partner on said nucleic acid array.

55-56. (canceled)

57. An in vitro method to analyse the development of cancerous cells from normal cells comprising
i) forming a preparation comprising a cell culture substrate according to claim 1 including cells;
ii) adding at least one agent capable of inducing cell transformation; and
iii) monitoring the effect, or not, of said agent on the transformation of said cells.

58. (canceled)

59. A process for the formation of a microcellular polymeric material comprising the steps of:
i) forming a preparation comprising an high internal phase emulsion comprising a hydrophobic elastomer at a concentration of between 20% (w/w) and 40% (w/w);
ii) forming a preparation comprising a catalyst;
iii) combining the preparations in (i) and (ii); and
iv) incubating the combined preparation to allow formation of a high internal phase emulsion polymer.

60-73. (canceled)

74. A high internal phase emulsion polymer obtained or obtainable by the process according to claim 59.

75. (canceled)

76. The use of a substrate comprising high internal phase emulsion polymer according to claim 1 to culture cells.

77-78. (canceled)

79. The use of a substrate comprising a high internal phase emulsion polymer according to claim 1 to determine the liver toxicity of an agent.

80-81. (canceled)

82. A method to test the liver toxicity of an agent comprising the steps of:
i) providing a cell culture comprising at least one hepatocyte cell and a cell culture substrate according to claim 1;
ii) adding at least one agent to be tested; and
iii) monitoring the activity of the agent with respect to the proliferation, differentiation or function of said hepatocyte cells as a measure of toxicity of the agent.

83-84. (canceled)

85. A method for the growth and differentiation of a keratinocyte and/a keratinocyte precursor stem cell comprising:
i) forming a preparation comprising a cell culture substrate according to claim 1, fibroblast feeder cells and cell culture medium;
ii) culturing said feeder cells to provide a cell culture substrate that is substantially coated with said feeder cells;
iii) contacting said coated substrate with keratinocytes and/or keratinocyte precursor stem cells; and
iv) culturing the combined cell preparation under conditions conducive to the growth and differentiation of said keratinocytes and/or keratinocyte precursor stein cells.

86-97. (canceled)

98. An apparatus for the culture of cells comprising a cell culture substrate according to claim 1, a cell culture vessel and an insert adapted to co-operate with said cell culture vessel and contain said cell culture substrate and said cells.

99. (canceled)

100. The use of a cell culture substrate according to claim 1 for the preparation of differentiated skin composite.
